(19) United States
(12) Patent Application Publication
(10) Pub. No.: US 2025/0257142 A1
(43) Pub. Date: Aug. 14, 2025

(54) ANTI-IL-31RA ANTIBODIES AND USES THEREOF

(71) Applicant: INVETX, INC., Boston, MA (US)

(72) Inventors: William Brondyk, Boston, MA (US); Leila Sevigny, Boston, MA (US)

(21) Appl. No.: 19/052,889

(22) Filed: Feb. 13, 2025

Related U.S. Application Data
(60) Provisional application No. 63/552,803, filed on Feb. 13, 2024.

(51) Int. Cl. C07K 16/28; G01N 33/68

(52) U.S. Cl. CPC C07K 16/2866; G01N 33/6854; C07K 2317/24; C07K 2317/76; C07K 2317/92; C07K 2317/94

(57) ABSTRACT

The invention provides novel anti-IL-31RA proteins, antibodies and IL-31RA binding fragments thereof, which inhibit association of IL-31 with IL-31 receptor and are suitable for administration to a human or canine subject. The invention provides novel compositions and methods of treating, alleviating the symptoms of, or preventing, allergic/inflammatory diseases, lung diseases, cardiovascular diseases, cancers, metabolic diseases, neurological diseases, and infectious diseases, comprising administering an effective amount of an anti-IL-31RA protein, antibody, or fragment thereof. The methods and compositions are used to treat or prevent IL-31-related disorders.

### INCORPORATION BY REFERENCE

This application claims priority to U.S. provisional application Ser. No. 63/552,803, filed Feb. 13, 2024, which is incorporated by reference herein in its entirety.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE LISTING

The instant application contains a Sequence Listing which has been submitted herewith and is hereby incorporated by reference in its entirety. Said .xml copy, created on Feb. 13, 2025 is named Y9432-01006, and is 153,457 bytes in size.

### FIELD OF THE INVENTION

The invention provides novel anti-IL-3IRA proteins and antibodies that are suitable for administration to a human or canine subject. The invention also provides novel compositions and methods of treating atopic dermatitis, dermatomyositis, pruritic skin disorders, allergic asthma, and inflammatory bowel diseases, primary localized cutaneous amyloidosis type 2, or eliciting an antiallergenic effect in a human or canine subject, comprising administering an effective amount of an anti-IL-3IRA protein, antibody or fragment thereof. The methods and compositions are used to treat or prevent IL-31-, IL-31-R-, and IL-31RA-related disorders.

### BACKGROUND OF THE INVENTION

Interleukin 31 (IL-31) is an inflammatory cytokine that helps trigger cell-mediated immunity against pathogens. Multiple cell types express IL-31 including activated Th2 cells, CD8+ T cells, skin-homing memory T cells, monocytes, macrophages, monocyte-derived dendritic cells, mast cells, keratinocytes, and dermal fibroblasts.

IL-31 is a member of the Interleukin 6 (IL-6) cytokine family, which also includes IL-6, IL-11, IL27 p28/IL-30, Leukemia inhibitory factor (LIF), Oncostatin M (OSM), Cardiotrophin-like cytokine (CLC), Ciliary neurotrophic factor (CNTF), Cardiotrophin-1 (CT-1), and Neuropoeitin. While IL-31 shares the four helical structure of the IL-6 family, IL-31 does not signal through the type I cytokine receptor gp130 receptor, which is shared between the IL-6, IL-11, IL-12, IL27, LIF, and OSM receptors. Instead, IL-31 signals through the IL-31 receptor (IL-31R), which is a heterodimeric receptor complex containing IL-31 Receptor Alpha (IL-31RA) and oncostatin M receptor (OSMR) beta chain. Although the OSMR beta chain is a subunit of the OSMR and IL-31R complexes, each has distinct biological function. IL-31 is involved in many Th2-driven diseases while OSM is involved in hematopoiesis and cancer development. IL-31R is expressed on macrophages, dendritic cells, eosinophils, basophils, keratinocytes, and peripheral nerves.

High levels of IL-31 are associated with conditions including pruritic skin disorders, allergic asthma, and inflammatory bowel diseases. IL-31 has also been shown to have chemokine-inducing activity by targeting immune cells such as mast cells, eosinophils, basophils, and monocytes/dendritic cells to induce inflammation. IL-31 has also been suggested to be involved in regulating the homeostasis of hematopoietic progenitor cells, and in the proliferation and differentiation of non-hematopoietic cells. IL-31 stimulates neuronal growth and sensory nerve branching. Within the skin, IL-31 impairs keratinocyte differentiation and barrier function, and in turn activates keratinocytes to produce cytokines, chemokines, and pruritus mediators amplifying skin inflammation and itch. IL-31 also interacts with dermal fibroblasts initiating tissue remodeling through the induction of collagen production and cytokine and chemokine expression.

In IL-31R-expressing sensory neurons, IL-31 induces the activation of ion channels (TRPV1, TRPA1) and transmits pruritus signals via brain natriuretic peptide to the central nervous system.

IL-31RA is expressed by multiple leukocyte subsets, and epithelial and stromal cells both in steady state and under activated conditions. For example, keratinoctyes, fibroblasts, and a distinct subset of dorsal root ganglia (DRG) neurons express and signal via IL-31RA. In macrophages, IL-4 and IL-13 upregulate the expression of IL-31RA.

IL-31RA has been found to cause primary localized cutaneous amyloidosis type 2 and implicated in pruritic skin disorders and allergic skin diseases such as atopic dermatitis. IL-31RA-related diseases and conditions that affect dogs, cats, and horses are similar to those in humans, including without limitation, atopic dermatitis, atopic eczema, lesional dermatomyositis, and other pruritic allergic skin diseases, and insect bite hypersensitivity.

Targeting IL-31 with nemolizumab appears efficacious in atopic dermatitis and prurigo nodularis. Similarly, targeting IL-31 with lokivetamab appears efficacious for the treatment of canine atopic dermatitis.

Citation or identification of any document in this application is not an admission that such document is available as prior art to the present invention.

### SUMMARY OF THE INVENTION

In an aspect, the invention provides an antigen binding protein that specifically binds to interleukin-31 receptor alpha (IL-31RA). In certain embodiments, the IL-3IRA binding protein comprises: (a) a heavy chain complementarity determining region 1 (HCDR1) comprising X₂₆X₂₇X₂₈X₂₉X₃₀X₃₁X₃₂X₃₃ (SEQ ID NO:53), wherein X₂₆ comprises A, G, I, L, M, W, F, P, or V, X₂₇ comprises A, G, I, L, M, W, F, P, V, or Y, X₂₈ comprises C, S, T, Y, N, or Q, X₂₉ comprises A, G, I, L, M, W, F, P, V, or Y, X₃₀ comprises D or E, X₃₁ comprises D or E, X₃₂ comprises C, S, T, Y, N, or Q, and X₃₃ comprises A, G, I, L, M, W, F, P, or V; (b) a heavy chain complementarity determining region 2 (HCDR2) comprising X₅₁X₅₂X₅₃X₅₄X₅₅X₅₆X₅₇ (SEQ ID NO:54), wherein X₅₁ comprises A, G, I, L, M, W, F, P, or V, X₅₂ comprises C, S, T, Y, N, or Q, X₅₃ comprises A, I, L, M, F, P, W, Y, or V, X₅₄ comprises A, G, I, L, M, W, F, P, or V, X₅₅ comprises A, G, I, L, M, W, F, P, or V, X₅₆ comprises A, G, I, L, M, W, F, P, or V, and X₅₇ comprises C, S, T, Y, N, or Q; (c) a heavy chain complementarity determining region 3 (HCDR3) comprising X₉₇X₉₈X₉₉X₁₀₀X₁₀₁X₁₀₂X₁₀₃X₁₀₄X₁₀₅X₁₀₆X₁₀₇X₁₀₈X₁₀₉X₁₁₀X₁₁₁X₁₁₂X₁₁₃X₁₁₄X₁₁₅X₁₁₆X₁₁₇X₁₁₈X₁₁₉ (SEQ ID NO:55), wherein X₉₇ comprises A, G, I, L, M, W, F, P, or V, X₉₈ comprises H, K, or R, X₉₉ comprises E or D, X₁₀₀ comprises C, S, T, Y, N, or Q, X₁₀₁ comprises A, G, I, L, M, W, F, P, or V, X₁₀₂ comprises A, G, I, L, M, W, F, P, or V, X₁₀₃ comprises H, K, or R, X₁₀₄ comprises A, G, I, L, M, W, F, P, or V, X₁₀₅ comprises A, G, I, L, M, W, F, P, or V, X₁₀₆ comprises E or D, X₁₀₇ comprises A, G, I, L, M, W, F, P, or V, X₁₀₈ comprises A, G, I, L, M, W, F, P, or V, X₁₀₉ comprises A, G, I, L, M, W, F, P, or V, X₁₁₀ comprises E or D, X₁₁₁ comprises C, S, T, Y, N, or Q, X₁₁₂ comprises A, G, I, L, M, W, F, P, V, or Y, X₁₁3 comprises an C, S, T, Y, N, or Q, X₁₁₄ comprises C, S, T, Y, N, or Q, X₁₁₅ comprises C, S, T, Y, N, or Q, X₁₁₆ comprises C, S, T, Y, N, or Q, X₁₁₇ comprises C, S, T, Y, N, or Q, X₁₁₈ comprises A, G, I, L, M, W, F, P, or V, and X₁₁₉ comprises E or D; (d) a light chain complementarity determining region 1 (LCDR1) comprising X₂₇X₂₈X₂₉X₃₀X₃₁X₃₂X₃₃ (SEQ ID NO:56), wherein X₂₇ comprises C, S, T, Y, N, or Q, X₂₈ comprises C, S, T, Y, N, or Q, X₂₉ comprises A, G, I, L, M, W, F, P, or V, X₃₀ comprises C, S, T, Y, N, or Q, X₃₁ comprises C, S, T, Y, N, or Q, X₃₂ comprises H, K, or R, and X₃₃ comprises C, S, T, Y, N, or Q; (e) a light chain complementarity determining region 2 (LCDR2) comprising X₅₁X₅₂X₅₃, wherein X₅₁ comprises A, G, I, L, M, W, F, P, or V, X₅₂ comprises C, S, T, Y, N, or Q, and X₅₃ comprises C, S, T, Y, N, or Q; and (f) a light chain complementarity determining region 3 (LCDR3) comprising X₉₀X₉₁X₉₂X₉₃X₉₄X₉₅X₉₆X₉₇X₉₈ (SEQ ID NO:57), wherein X₉₀ comprises C, S, T, Y, N, or Q, X₉₁ comprises C, S, T, Y, N, or Q, X₉₂ comprises C, S, T, Y, N, or Q, X₉₃ comprises E or D, X₉₄ comprises C, S, T, Y, N, or Q, X₉₅ comprises C, L, S, T, Y, N, or Q, X₉₆ comprises A, G, I, L, M, W, F, P, or V, X₉₇ comprises H, K, or, R, and X₉₈ comprises C, S, T, Y, N, or Q.

In certain embodiments, (a) HCDR1 comprises GFTFDDYG (SEQ ID NO:29) or differs at no more than one or two positions; and/or (b) HCDR2 comprises INX₅₃AGGT (SEQ ID NO:58), wherein X₅₃ comprises F, W, or Y; and/or (c) HCDR3 comprises ARESPLRLGDLGGDYFYYYYYX₁₁₈D (SEQ ID NO:59), wherein X₁₁₈ comprises I, L, or M; and/or (d) LCDR1 comprises QSVSSRY (SEQ ID NO:32) or differs at no more than one or two positions; and/or (e) LCDR2 comprises GTS or differs at no more than one or two positions; and/or (f) LCDR3 comprises QQYDX₉₄X₉₅PRT (SEQ ID NO:60), wherein X₉₄ comprises N, or Q, and X₉₅ comprises S or L.

In certain embodiments, the heavy chain variable domain comprises W53F, W53Y, M118I, or M118L and/or the light chain variable domain comprises N94Q or S95L.

In certain embodiments, the heavy chain variable domain comprises an HCDR2 sequence, an HCDR3 sequence, and an LCDR3 sequence of Table 6.

In certain embodiments, the heavy chain variable domain comprises the IMGT CDRs of SEQ ID NO:1, SEQ ID NO:10, SEQ ID NO:14, SEQ ID NO:15, SEQ ID NO:16, or SEQ ID NO:17, and wherein the light chain variable domain comprises the CDRs of SEQ ID NO:2, SEQ ID NO:11, SEQ ID NO:19, or SEQ ID NO:20.

In certain embodiments, antigen binding protein that specifically binds to IL-3IRA comprises: (a) a heavy chain complementarity determining region 1 (HCDR1) comprising X₂₆X₂₇X₂₈X₂₉X₃₀X₃₁X₃₂X₃₃ (SEQ ID NO:61), wherein X₂₆ comprises A, G, I, L, M, W, F, P, or V, X₂₇ comprises A, G, I, L, M, W, F, P, V, or Y, X₂₈ comprises C, S, T, Y, N, or Q, X₂₉ comprises A, G, I, L, M, W, F, P, V, of Y, X₃₀ comprises C, S, T, Y, N, or Q, X₃₁ comprises C, S, T, Y, N, or Q, X₃₂ comprises C, S, T, Y, N, or Q, and X₃₃ comprises A, G, I, L, M, W, F, P, or V; (b) a heavy chain complementarity determining region 2 (HCDR2) comprising X₅₁X₅₂X₅₃X₅₄X₅₅X₅₆X₅₇ (SEQ ID NO:62), wherein X₅₁ comprises A, G, I, L, M, W, F, P, or V, X₅₂ comprises C, S, T, Y, N, or Q, X₅₃ comprises A, G, I, L, M, W, F, P, or V, X₅₄ comprises C, S, T, Y, N, or Q, X₅₅ comprises A, G, I, L, M, W, F, P, or V, X₅₆ comprises C, S, T, Y, N, or Q, and X₅₇ comprises A, C, S, T, Y, N, or Q; (c) a heavy chain complementarity determining region 3 (HCDR3) comprising X₉₇X₉₈X₉₉X₁₀₀X₁₀₁X₁₀₂X₁₀₃X₁₀₄X₁₀₅X₁₀₆X₁₀₇ (SEQ ID NO:63), wherein X₉₇ comprises A, G, I, L, M, W, F, P, or V, X₉₈ comprises C, S, T, Y, N, or Q, X₉₉ comprises C, S, T, Y, N, or Q, X₁₀₀ comprises A, G, I, L, M, W, F, P, or V, X₁₀₁ comprises A, G, I, L, M, W, F, P, or V, X₁₀₂ comprises C, S, T, Y, N, or Q, X₁₀₃ comprises C, S, T, Y, N, or Q, X₁₀₄ comprises A, G, I, L, M, W, F, P, V, or Y, X₁₀₅ comprises H, K, or R, X₁₀₆ comprises A, G, I, L, M, W, F, P, or V, and X₁₀₇ comprises E or D; (d) a light chain complementarity determining region 1 (LCDR1) comprising X₂₇X₂₈X₂₉X₃₀X₃₁X₃₂ (SEQ ID NO:64) wherein X₂₇ comprises C, S, T, Y, N, or Q, X₂₈ comprises C, S, T, Y, N, or Q, X₂₉ comprises A, G, I, L, M, V, W, F, P, or V, X₃₀ comprises C, S, T, Y, N, or Q, X₃₁ comprises C, S, T, Y, N, or Q, and X₃₂ comprises C, S, T, Y, N, or Q; (e) a light chain complementarity determining region 2 (LCDR2) comprising X₅₀X₅₁X₅₂, wherein X₅₀ comprises A, G, I, L, M, W, F, P, or V, X₅₁ comprises A, G, I, L, M, W, F, P, or V, and X₅₂ comprises C, S, T, Y, N, or Q; and (f) a light chain complementarity determining region 3 (LCDR3) comprising X₈₉X₉₀X₉₁X₉₂X₉₃X₉₄X₉₅X₉₆X₉₇X₉₈ (SEQ ID NO:65), wherein X₈₉ comprises C, S, T, Y, N, or Q, X₉₀ comprises C, S, T, Y, N, or Q, X₉₁ comprises A, G, I, L, M, W, F, P, V, or Y, X₉₂ comprises A, G, I, L, M, W, F, P, V, or Y, X₉₃ comprises C, S, T, Y, N, or Q, X₉₄ comprises A, I, L, M, F, P, W, Y, or V, X₉₅ comprises A, G, I, L, M, W, F, P, or V, X₉₆ comprises A, G, I, L, M, W, F, P, or V, X₉₇ comprises A, G, I, L, M, W, F, P, V, or Y, and X₉₈ comprises C, S, T, Y, N, or Q.

In certain embodiments, (a) HCDR1 comprises GFTFSSYA (SEQ ID NO:34); and/or (b) HCDR2 comprise ISGSGX₅₆X₅₇T (SEQ ID NO:66), wherein X₅₆ comprises N, or Q; and X₅₇ comprises A or S; and/or (c) HCDR3 comprises ATQVVYYFKX₁₀₆DV (SEQ ID NO:67), wherein X₁₀₆ comprises I, L, or M; and/or (d) LCDR1 comprises QSVSSN (SEQ ID NO:37); and/or (e) LCDR2 comprises GAS; and/or (f) LCDR3 comprises QQYYNX₉₄PPFT (SEQ ID NO:68), wherein X₉₄ comprises F, Y, or W.

In certain embodiments, the heavy chain variable domain comprises N56Q, S57A, M106I, or M1106L and/or the light chain variable domain comprises W94F or W94Y.

In certain embodiments, the heavy chain variable domain comprises an HCDR2 sequence, an HCDR3 sequence, and an LCDR3 sequence of Table 7.

In certain embodiments, the heavy chain variable domain comprises the IMGT CDRs of SEQ ID NO:3, SEQ ID NO:12, SEQ ID NO:21, SEQ ID NO:22, SEQ ID NO:23, SEQ ID NO:24, or SEQ IDNO:25, and wherein the light chain variable domain comprises the CDRs of SEQ ID NO:4, SEQ ID NO:13, SEQ ID NO:26, or SEQ ID NO:27.

In certain embodiments, the antigen binding protein comprises a heavy chain variable domain at least 80%, at least 85%, at least 90%, at least 95%, at least 97%, at least 98%, at least 99%, or 100% identical to a heavy chain variable domain of SEQ ID NO:3, SEQ ID NO:12, SEQ ID NO:21, SEQ ID NO:22, SEQ ID NO:23, SEQ ID NO:24, or SEQ IDNO:25, and a light chain variable domain at least 80%, at least 85%, at least 90%, at least 95%, at least 97%, at least 98%, at least 99%, or 100% identical to a light chain variable domain of SEQ ID NO:4, SEQ ID NO:13, SEQ ID NO:26, or SEQ ID NO:27.

In an aspect, the invention provides an isolated nucleic acid sequence encoding any one of the aforementioned anti-IL-3IRA antibodies or antibody fragments, and a vector comprising or capable of expressing any one of the anti-IL-3IRA antibodies or antibody fragments.

In another aspect, the invention provides a recombinant cell which comprises a nucleic acid sequence encoding any one of the aforementioned anti-IL-31RA antibodies or antibody fragments, or a vector comprising or capable of expressing any one of the anti-IL-31RA antibodies or antibody fragments.

The invention provides a method of producing any one of the aforementioned anti-IL-31RA antibodies or antibody fragments, which comprises culturing the cell capable of expressing the anti-IL-3IRA antibody or antibody fragment under conditions that result in production of the antibody or antibody fragment.

The invention provides a pharmaceutical composition comprising a therapeutically effective amount of any one of the aforementioned anti-IL-31RA antibodies or antibody fragments.

In an aspect, the invention provides a method of inhibiting binding of IL-31 to IL-3IRA in a subject, which comprises administering to the subject a therapeutically effective amount of any one of the aforementioned anti-IL-3IRA antibodies or antibody fragments.

In an aspect, the invention provides a method of suppressing atopic dermatitis in a subject, which comprises administering to the subject a therapeutically effective amount of any one of the aforementioned anti-IL-3IRA antibodies or antibody fragments.

In certain non-limiting embodiments, the subject is a human, a canine, a feline or an equine.

In an aspect, the invention provides a method of blocking binding of IL-31 to IL-3IRA in a sample comprising incubating the sample with any one of the aforementioned anti-IL-3IRA antibodies or antibody fragments and detecting whether IL-31 binds to IL-31RA.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 depicts the human antibody inhibition of IL-31-induced STAT3 phosphorylation in DH82 cells.

FIG. 2 depicts the caninized 109 antibody inhibition of IL-31-induced STAT3 phosphorylation in DH82 cells.

FIG. 3 depicts the caninized 186 antibody inhibition of IL-31-induced STAT3 phosphorylation in DH82 cells.

FIG. 4 depicts the inhibition of IL-31-induced STAT3 phosphorylation in DH82 cells by optimized, caninized antibodies.

FIG. 5 (SEQ ID Nos:1,10,14,15,16,17,18,29,82-97) depicts a sequence alignment of heavy chain variable domains of the caninized 109 antibody.

FIG. 6 (SEQ ID Nos:2,11,19,20,32-33,43,44,98-104) depicts a sequence alignment of light chain variable domains of the caninized 109 antibody.

FIG. 7 (SEQ ID Nos:3,12,21,22,23,24,25,34-36,45-48,83,96-97,105-109) depicts a sequence alignment of heavy chain variable domains of the caninized 186 antibody.

FIG. 8 (SEQ ID Nos:4,13,26,27,37-38,49-50,110-117) depicts a sequence alignment of light chain variable domains of the caninized 186 antibody.

### DETAILED DESCRIPTION OF THE INVENTION

According to certain exemplary embodiments of the present invention, the IL-3IRA binding protein is an anti-IL-31RA antibody or antigen-binding fragment thereof. The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In different embodiments of the invention, the FRs of the antibody (or antigen-binding portion thereof) may be identical to the canine germline sequences, or may be naturally or artificially modified. An amino acid consensus sequence may be defined based on a side-by-side analysis of two or more CDRs.

Antibody residues that have a substantial impact on affinity and specificity of binding to target antigen are primarily located in CDRs. Kabat et al. compiled and aligned immunoglobulin heavy and light chain sequences and were the first to propose a standardized numbering scheme for the variable regions of immunoglobulins identifying conserved and hypervariable regions and residues. (Kabat E A et al., 1979, Sequences of Immunoglobulin Chains: Tabulation and Analysis of Amino Acid Sequences of Precursors, V-regions, C-regions, J-Chain and BP-Microglobulins, Department of Health, Education, and Welfare, Public Health Service, National Institutes of Health). While the Kabat system is a widely adopted standard for numbering antibody residues, the hypervariable regions defined by Kabat do not exactly match with the structural aspects of antigen-binding loops. Chothia and Lesk developed a structure-based numbering scheme by aligning crystal structures of antibody variable regions and classified CDR loops in a small number of “canonical” classes (Chothia C, et al., 1987, Canonical structures for the hypervariable regions of immunoglobulins. J. Mol. Biol. 196:901-17. doi: 10.1016/0022-2836(87)90412-8). An advantage of the Chothia numbering scheme is that topologically aligned residues from different antibodies are localized at the same position number and the Chothia CDR definition corresponds in most antibody sequences to the structural antigen-binding loop. Lefranc introduced a new system based on germ-line sequences intended to standardize numbering for all proteins of the immunoglobulin superfamily, including T cell receptor chains. (Giudicelli V et al., 1997, IMGT, the international ImMunoGeneTics database. Nucleic Acids Res. 25:206-11), which was then extended to entire variable domains (Lefranc M-P et al., 2003, IMGT unique numbering for immunoglobulin and T cell receptor variable domains and Ig superfamily V-like domains. Dev Comp Immunol. 27:55-77. doi: 10.1016/S0145-305X(02)00039-3). Additional numbering systems have been proposed to align unconventional frameworks (Abhinandan K R et al., 2008, Analysis and improvements to Kabat and structurally correct numbering of antibody variable domains. Mol Immunol. 45:3832-9. doi: 10.1016/j.molimm.2008.05.022) and to subdivide variable chain sequences into multiple fragments including structurally invariant “cores” (Gelfand et al., 1998, Algorithmic determination of core positions in the VL and VH domains of immunoglobulin molecules. J Comput Biol. (1998) 5:467-77). In certain embodiments of the invention, CDR residues are identified according to such a standard system as set forth above. In certain embodiments, antibodies of the invention are identified by all or a subset of Kabat CDR residues of the antibody sequences set forth herein. In certain embodiments, antibodies of the invention are identified by all or a subset of Chothia CDR residues of the antibody sequences set forth herein. In certain embodiments, antibodies of the invention are identified by all or a subset of IMGT CDR residues of the antibody sequences set forth herein. In certain embodiments, antibodies of the invention are identified by CDR residues defined by two or more systems, comprising e.g., but not limited to, all or a subset of residues of amino acids of HCDR1 according to a first system, e.g. Kabat, all or a subset of residues of HCDR2 according to a second system, e.g. Chothia, all or a subset of residues of HCDR3 according to Kabat, all or a subset of residues of LCDR1 according to Kabat, all or a subset of residues of LCDR2 according to IMGT, and all or a subset of residues of LCDR3 according to Chothia. Such is just one example of identifying CDR amino acids according to more than one system. In certain embodiments, CDRs may be most conveniently or most accurately described to include amino acids selected by the Applicant. According to the invention, CDRs can be described or identified based on amino acids observed to strongly determine antigen biding and amino acids observed not to participate in antigen binding. Framework residues are those flanking the CDRs.

For reference, the table below shows relative locations of Kabat, Chothia, and IMGT CDRs mapped on the antigen binding regions of caninized antibody 109-49 (V_{H}: SEQ ID NO:10; V_{L} SEQ ID NO:11) and on caninized antibody 186-95 (V_{H}: SEQ ID NO:12; V_{L}: SEQ ID NO:13) described herein that bind to canine IL-31RA. “X” represents amino acid positions starting from the amino terminal of the antibody V_{H} or V_{L} chains as follows: 109-49-V_{H}: X₂₅X₂₆X₂₇X₂₈X₂₉X₃₀X₃₁X₃₂X₃₃X₃₄X₃₅X₃₆ represents the positions of SGFTFDDYGMSW (SEQ ID NO:69) in the region of CDR1H, X₄₉X₅₀X₅₁X₅₂X₅₃X₅₄X₅₅X₅₆X₅₇X₅₈X₅₉X₆₀X₆₁X₆₂X₆₃X₆₄X₆₅X₆₆X₆₇ represents the positions of SGINWAGGTIGYADAVKGR (SEQ ID NO:70) in the region of CDR2H, and X₉₆X₉₇X₉₈X₉₉X₁₀₀X₁₀₁X₁₀₂X₁₀₃X₁₀₄X₁₀₅X₁₀₆X₁₀₇X₁₀₈X₁₀₉X₁₁₀X₁₁X₁₁₂X₁₁₃X₁₁₄X₁₁₅X₁₁₆X₁₁₇X₁₁₈X₁₁₉X₁₂₀X₁₂₁ represents the positions of CARESPLRLGDLGGDYFYYYYYMDVW (SEQ ID NO:71) in the region of CDR3H; 109-49-V_{L}: X₂₃X₂₄X₂₅X₂₆X₂₇X₂₈X₂₉X₃₀X₃₁X₃₂X₃₃X₃₄X₃₅X₃₆ represents the positions of CRASQSVSSRYLVW (SEQ ID NO:72) in the region of CDR1L, X₅₀X₅₁X₅₂X₅₃X₅₄X₅₅X₅₆X₅₇X₅₈ represents the positions of YGTSSRATG (SEQ ID NO:73) in the region of CDR2L, and X₈₉X₉₀X₉₁X₉₂X₉₃X₉₄X₉₅X₉₆X₉₇X₉₈X₉₉ represents the positions of CQQYDNSPRTF (SEQ ID NO:74) in the region of CDR3L.

186-95-V_{H}: X₂₅X₂₆X₂₇X₂₈X₂₉X₃₀X₃₁X₃₂X₃₃X₃₄X₃₅X₃₆ represents the positions of SGFTFSSYAMSW (SEQ ID NO:80) in the region of CDR1H, X₄₉X₅₀X₅₁X₅₂X₅₃X₅₄X₅₅X₅₆X₅₇X₅₈X₅₉X₆₀X₆₁X₆₂X₆₃X₆₄X₆₅X₆₆X₆₇ represents the positions of SAISGSGNSTYYADAVKGR (SEQ ID NO:75) in the region of CDR2H, and X₉₆X₉₇X₉₈X₉₉X₁₀₀X₁₀₁X₁₀₂X₁₀₃X₁₀₄X₁₀₅X₁₀₆X₁₀₇X₁₀₈X₁₀₉ represents the positions of CATQVVYYFKMDVW (SEQ ID NO:76) in the region of CDR3H; 186-95-V_{L}: X₂₃X₂₄X₂₅X₂₆X₂₇X₂₈X₂₉X₃₀X₃₁X₃₂X₃₃X₃₄X₃₅ represents the positions of CKSSQSVSSNLAW (SEQ ID NO:77) in the region of CDR1L, X₄₉X₅₀X₅₁X₅₂X₅₃X₅₄X₅₅X₅₆X₅₇ represents the positions of YGASTRASG (SEQ ID NO:78) in the region of CDR2L, and X₈₈X₈₉X₉₀X₉₁X₉₂X₉₃X₉₄X₉₅X₉₆X₉₇X₉₈X₉₉ represents the positions of CQQYYNWPPFTF (SEQ ID NO:79) in the region of CDR3L.

| | CDR1H | CDR2H |
|---|---|---|
| Kabat | XXXXX | XXXXXXXXXXXXXXXXX |
| Chothia | XXXXXXX | XXXXXX |
| IMGT | XXXXXXXX | XXXXXXXX |
| 109-49 (SEQ ID NO: 10) | SGFTFDDYGMSW | SGINWAGGTIGYADAVKGR |
| | (SEQ ID NO: 69) | (SEQ ID NO: 70) |
| 186-95 (SEQ ID NO: 12) | SGFTFSSYAMSW | SAISGSGNSTYYADAVKGR |
| | (SEQ ID NO: 80) | (SEQ ID NO: 75) |
| | CDR3H | |
| Kabat | XXXXXXXXXXXXXXXXXXXXXX | |
| Chothia | XXXXXXXXXXXXXXXXXXXXXX | |
| IMGT | XXXXXXXXXXXXXXXXXXXXXXXX | |
| 109-49 (SEQ ID NO: 10) | CARESPLRLGDLGGDYFYYYYYMDVW | |
| | (SEQ ID NO: 71) | |
| 186-95 (SEQ ID NO: 12) | CATQ------------VVYYFKMDVW | |
| | (SEQ ID NO: 76) | |
| | CDR1L | CDR2L |
| Kabat | XXXXXXXXXXXX | XXXXXXX |
| Chothia | XXXXXXXXXXXX | XXXXXXX |
| IMGT | XXXXXXX | XXX |
| 109-49 (SEQ ID NO: 11) | CRASQSVSSRYLVW | YGTSSRATG |
| | (SEQ ID NO: 72) | (SEQ ID NO: 73) |
| 186-95 (SEQ ID NO: 13) | CKSSQSVSSN-LAW | YGASTRASG |
| | (SEQ ID NO: 77) | (SEQ ID NO:78) |

| | CDR3L |
|---|---|
| Kabat | XXXXXXXXXX |
| Chothia | XXXXXXXXXX |
| IMGT | XXXXXXXXXX |
| 109-49 (SEQ ID NO: 11) | COQYDN-SPRTF (SEQ ID NO: 74) |
| 186-95 (SEQ ID NO: 13) | CQQYYNWPPFTF (SEQ ID NO: 79) |

In one aspect, the invention provides a binding protein suitable for use in a mammal, for example, without limitation, a canine. In certain embodiments, a caninized anti-IL-31RA binding protein comprises a heavy chain complementarity determining region 1 (CDR1H), a heavy chain complementarity determining region 2 (CDR2H), a heavy chain complementarity determining region 3 (CDR3H), a light chain complementarity determining region 1 (CDR1L), a light chain complementarity region 2 (CDR2L), and a light chain complementarity region 3 (CDR3L). The CDRs may be designated according to Kabat, Chothia, IMGT, a combination thereof, or as otherwise set forth herein.

According to the invention, antibodies are characterized by CDRs. In embodiments where variable domain CDRs are defined according to IMGT, Kabat, or Chothia systems, the variable domain frameworks comprise the amino acids not included in the CDRs. Likewise, in embodiments where variable domain CDRs are identified by amino acid position, the variable domain frameworks comprise the amino acids not included in the CDRs.

In certain embodiments, the anti-IL-3IRA binding protein comprises one or more (i.e. one, two, three, four, five, or all six) CDRs of a caninized anti-IL-3IRA binding protein set forth herein. In certain embodiments, the anti-IL-3IRA binding protein comprises one or more (i.e. one, two, three, four, five, or all six) CDRs of an affinity matured caninized antibody disclosed herein. Such CDRs may further comprise amino acid changes at one or two positions, for example incorporating an amino acid demonstrated herein to be compatible with IL-31RA binding or a conservative substitution.

In certain embodiments, a binding protein of the invention comprises a caninized antibody or is suitable for administration to a canine. In certain embodiments, a binding proteins of the invention comprises a felinized antibody or is suitable for administration to a feline. In certain embodiments, a binding protein of the invention comprises an equinized antibody or is suitable for administration to an equine. In certain embodiments, a binding proteins of the invention comprises a humanized antibody or is suitable for administration to a human.

In certain embodiments, an amino acid residue is mutated into one that allows the properties of the amino acid side chain to be conserved. Examples of the properties of amino acid side chains comprise: polar amino acids (C, S, T, Y, N, Q), nonpolar amino acids (A, G, I, L, M, W, F, P, V), basic amino acids (H, K, R), acidic amino acids (E, D), hydrophobic amino acids (A, I, L, M, F, P, W, Y, V), hydrophilic amino acids (R, D, N, C, E, Q, G, H, K, S, T), and amino acids comprising the following side chains: aliphatic side-chains (G, A, V, L, I, P); hydroxyl group-containing side-chains (S, T, Y); sulfur atom-containing side-chains (C, M); carboxylic acid- and amide-containing side-chains (D, N, E, Q); base-containing side-chains (R, K, H); and aromatic-containing side-chains (H, F, Y, W). The letters within parenthesis indicate the one-letter amino acid codes. Amino acid substitutions within each group are called conservative substitutions. It is well known that a polypeptide comprising a modified amino acid sequence in which one or more amino acid residues is deleted, added, and/or substituted can retain the original biological activity (Mark D. F. et al., Proc. Natl. Acad. Sci. U.S.A. 81:5662-5666 (1984); Zoller M. J. and Smith M., Nucleic Acids Res. 10: 6487-6500 (1982); Wang A. et al., Science 224: 1431-1433; Dalbadie-McFarland G. et al., Proc. Natl. Acad. Sci. U.S.A. 79: 6409-6413 (1982)). The number of mutated amino acids is not limited, but in general, the number falls within 40% of amino acids of each CDR, and preferably within 35%, and still more preferably within 30% (e.g., within 25%). The identity of amino acid sequences can be determined as described herein.

The invention provides recombinant antibodies designed or modified to minimize immunogenicity when administered to a subject, for example, without limitation, chimerization, caniniation, felinization, or humanization. In certain embodiments, the antibodies are further modified to remove T cell epitopes. Without limitation, the subject can be a canine or feline. The subject can be a human or a non-human primate. The subject can be a farm animal.

As used herein, the term “canine” refers to any member of the Canidae family. Domestic dogs, pure-bred and/or mongrel companion dogs, and wild or feral dogs are all canines.

As used herein the term “human framework” or “canine framework” refers to the amino acid sequence of the heavy chain and light chain of a canine antibody other than the hypervariable region residues defined herein as CDR residues. With regard to a humanized antibody, in certain embodiments, canine CDRs are identified in human antibody heavy and light chains variable domain sequences that closely match CDRs of IL-31RA-binding antibodies originating in other species. In certain embodiments, native human CDRs are replaced with the corresponding foreign CDRs (e.g., those from a rat or a mouse antibody) in both chains. With regard to a caninized antibody, in certain embodiments, canine CDRs are identified in canine antibody heavy and light chains variable domain sequences that closely match CDRs of IL-31RA-binding antibodies originating in other species. In certain embodiments, native canine CDRs are replaced with the corresponding foreign CDRs (e.g., those from a rat or a mouse antibody) in both chains. Optionally the heavy and/or light chains of the humanized or caninized antibody may contain some mutated or foreign non-CDR residues, e.g., framework amino acid residues that vary among germline antibody sequence or mutations that preserve the conformation of the foreign CDRs within the antibody.

Five major isotypes (IgA, IgG, IgM, IgD, IgE) and two forms of light chain (κ and λ) are present in dogs. In the dog, there are four subtypes for IgG, which are IgGA, IgGB, IgGC, and IgGD (Bergeron etal al, 2014, Comparative functional characterization of canine IgG subclasses. Veterinary Immunology and Immunopathology. 157:31-41). For the cat, there are three subtypes of IgG which are IgG1a, IgG1b, and IgG2 (Streitzel et al. 2014, In vitro functional characterization of canine IgGs. Vet Immunol Immunopathol 158, 214-223, doi.org/10.1016/j.vetimm.2014.01.012).

In certain embodiments, antibodies of the invention, including but not limited to caninized, felinized, and humanized antibodies, are engineered to modulate one or more effector functions or circulation half-life. Hinge and constant domains of an antibody engage host receptors or complement protein to mediate effector functions and regulate antibody circulation. In certain embodiments, one or more effector functions is enhanced. In certain embodiments, one or more effector functions is reduced or eliminated. In certain embodiments, antibodies of the invention comprise modifications to modulate antibody-dependent cytotoxicity (ADCC) and/or complement-dependent cytotoxicity (CDC). A non-limiting example involves engineering of canine or feline constant region residues Met234 and/or Leu235 (EU numbering) to reduce effector function (see, e.g., Lund et al., Human Fc gamma RI and Fc gamma RII interact with distinct but overlapping sites on human IgG. J Immunol., 1991, 147:2657-62). In certain embodiments, a constant region of a canine or feline or other antibody comprises 234A and L235A substitutions. In certain embodiments, the second constant domain (CH2) and/or the third constant domain (CH3) comprises mutations and combinations of mutations compared to wild-type designed to modulate binding to FcRn (neonatal Fc) receptor. In canine constant regions, such mutations include, without limitation substitutions of Ala426, for example A426Y or A426H, substitutions of Thr286, for example T286L or T286Y, substitutions of Tyr436, for example Y436H, and combinations of such mutations including but not limited to A426Y+T286L, A426Y+Y436H, A426H+T286L, and A426H+T286Y. In certain embodiments a chimeric or caninized antibody of the invention comprises a substitution at amino acid Asn434, such as but not limited to N434H. In feline constant regions, such mutations include, without limitation substitutions of Ser428, including but not limited to S428Y or S428L, substitutions of Gln311, including but not limited to Q311V, substitutions of Leu309, including but not limited to L309V, substitutions of Thr286, including but not limited to T286E, substitutions of Glu380, including but not limited to E380T, and combinations of such mutations including but not limited to S428Y+Q311V, S428Y+L309V, S428Y+Q311V+T286E, S428Y+Q311V+E380T, and S428Y+L309V+E380T. In certain embodiments a chimeric or felinized antibody of the invention comprises a substitution at amino acid Ser428 and/or Ser434 including but not limited to S428L and/or S434H. WO 2021/231464 describes mutations of canine constant regions for modulating binding affinity to FcRn, including but not limited to T286L and A426Y (EU numbering). WO 2022/125355 likewise describes mutations of constant regions of livestock animals.

The term “antibody,” as used herein, includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. As used herein, the term “specifically binds” or “binds specifically” means that an IL-3IRA binding protein of the invention reacts or associates more frequently, more rapidly, with greater duration and/or with greater affinity with IL-3IRA than it does with alternative antigens. For example, IL-31RA binding protein binds to IL-31RA with materially greater affinity (e.g., at least 2-fold or 5-fold or 10-fold or 20-fold or 50-fold or 100-fold or 500-fold or 1000-fold or 10,000-fold or greater) than it does to other proteins or peptides. In certain embodiments, the IL-31RA-binding proteins binds to IL-31RA with an equilibrium dissociation constant KD for the epitope or target to which it binds of, e.g., 10⁴ M or smaller, e.g., 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. It will be recognized by one of skill that an antibody that specifically binds to a target (e.g., IL-31RA) from one species may also specifically bind to orthologs of IL-31RA.

Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

In certain embodiments, an antigen-binding fragment of an antibody comprises at least one variable domain. The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) VH-CH1; (ii) VH-CH2; (iii) VH-CH3; (iv) VH-CH1-CH2; (V) VH-CH1-CH2-CH3; (vi) VH-CH2-CH3; (vii) VH-CL; (viii) VL-CH1; (ix) VL-CH2, (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homodimer or hetero-dimer (or other multimer) of any of the variable and constant domain configurations listed above in non-covalent association with one another and/or with one or more monomeric VH or VL domain (e.g., by disulfide bond(s)).

The term “diabody (Db)” refers to a bivalent antibody fragment constructed by gene fusion (for example, P. Holliger et al., Proc. Natl. Acad. Sci. USA 90: 6444-6448 (1993), EP 404,097, WO 93/11161). In general, a diabody is a dimer of two polypeptide chains. In the each of the polypeptide chains, a light chain variable region (VL) and a heavy chain variable region (VH) in an identical chain are connected via a short linker, for example, a linker of about five residues, so that they cannot bind together. Because the linker between the two is too short, the VL and VH in the same polypeptide chain cannot form a single chain V region fragment, but instead form a dimer. Thus, a diabody has two antigen-binding domains. When the VL and VH regions against the two types of antigens (a and b) are combined to form VLa-VHb and VLb-VHa via a linker of about five residues, and then co-expressed, they are secreted as bispecific Dbs. The antibodies of the present invention may be such Dbs.

A single-chain antibody (also referred to as “scFv”) can be prepared by linking a heavy chain V region and a light chain V region of an antibody (for a review of scFv see Pluckthun “The Pharmacology of Monoclonal Antibodies” Vol. 113, eds. Rosenburg and Moore, Springer Verlag, N.Y., pp. 269-315 (1994)). Methods for preparing single-chain antibodies are known in the art (see, for example, U.S. Pat. Nos. 4,946,778; 5,260,203; 5,091,513; and 5,455,030). In such scFvs, the heavy chain V region and the light chain V region are linked together via a linker, preferably, a polypeptide linker (Huston, J. S. et al., Proc. Natl. Acad. Sci. U.S.A., 1988, 85, 5879-5883). The heavy chain V region and the light chain V region in a scFv may be derived from the same antibody, or from different antibodies. The peptide linker used to ligate the V regions may be any single-chain peptide consisting of 12 to 19 residues. A DNA encoding a scFv can be amplified by PCR using, as a template, either the entire DNA, or a partial DNA encoding a desired amino acid sequence, selected from a DNA encoding the heavy chain or the V region of the heavy chain of the above antibody, and a DNA encoding the light chain or the V region of the light chain of the above antibody; and using a primer pair that defines the two ends. Further amplification can be subsequently conducted using a combination of the DNA encoding the peptide linker portion, and the primer pair that defines both ends of the DNA to be ligated to the heavy and light chain respectively. After constructing DNAs encoding scFvs, conventional methods can be used to obtain expression vectors comprising these DNAs, and hosts transformed by these expression vectors. Furthermore, scFvs can be obtained according to conventional methods using the resulting hosts. These antibody fragments can be produced in hosts by obtaining genes that encode the antibody fragments and expressing these as outlined above. Antibodies bound to various types of molecules, such as polyethylene glycols (PEGs), may be used as modified antibodies. Methods for modifying antibodies are already established in the art. The term “antibody” in the present invention also encompasses the above-described antibodies.

The term “Kd” as used herein, refers to the dissociation constant of an antibody-antigen interaction. The dissociation constant, Kd, and the association constant, Ka, are quantitative measures of affinity. At equilibrium, free antigen (Ag) and free antibody (Ab) are in equilibrium with antigen-antibody complex (Ag-Ab), and the rate constants, ka and kd, quantitate the rates of the individual reactions. At equilibrium, ka [Ab][Ag]=kd [Ag−Ab]. The dissociation constant, Kd, is given by: Kd=kd/ka=[Ag][Ab]/[Ag−Ab]. Kd has units of concentration, most typically M, mM, nM, pM, etc. When comparing antibody affinities expressed as Kd, having greater affinity for IL-31RA is indicated by a lower value. The association constant, Ka, is given by: Ka=ka/kd=[Ag−Ab]/[Ag][Ab]. Ka has units of inverse concentration, most typically M-1, mM-1, nM-1, pM-1, etc. As used herein, the term “avidity” refers to the strength of the antigen-antibody binding taking valency into account.

The antibodies obtained can be purified to homogeneity. The antibodies can be isolated and purified by a method routinely used to isolate and purify proteins. The antibodies can be isolated and purified by the combined use of one or more methods appropriately selected from column chromatography, filtration, ultrafiltration, salting out, dialysis, preparative polyacrylamide gel electrophoresis, and isoelectro-focusing, for example (Strategies for Protein Purification and Characterization: A Laboratory Course Manual, Daniel R. Marshak et al. eds., Cold Spring Harbor Laboratory Press (1996); Antibodies: A Laboratory Manual. Ed Harlow and David Lane, Cold Spring Harbor Laboratory, 1988). Such methods are not limited to those listed above. Chromatographic methods include affinity chromatography, ion exchange chromatography, hydrophobic chromatography, gel filtration, reverse-phase chromatography, and adsorption chromatography. These chromatographic methods can be practiced using liquid phase chromatography, such as HPLC and FPLC. Columns to be used in affinity chromatography include protein A columns and protein G columns. For example, protein A columns include Hyper D, POROS, and Sepharose F. F. (Pharmacia). Antibodies can also be purified by utilizing antigen binding, using carriers on which antigens have been immobilized.

As used herein, the term “therapeutic agent” refers to any agent or material that has a beneficial effect on the mammalian recipient. Thus, “therapeutic agent” embraces both therapeutic and prophylactic molecules having nucleic acid or protein components.

“Treating” as used herein refers to ameliorating at least one symptom of, curing and/or preventing the development of a given disease or condition.

The anti-IL-3IRA proteins described herein, including antibodies or fragments thereof, are useful for ameliorating, or reducing the symptoms of, or treating, or preventing, diseases or conditions associated, characterized by, or caused by altered levels of IL-31. Such diseases or conditions include, without limitation, atopic dermatitis, dermatomyositis, pruritic skin disorders, allergic asthma, inflammatory bowel diseases, primary localized cutaneous amyloidosis type 2, atopic eczema, lesional dermatomyositis, and insect bite hypersensitivity. The anti-IL-31RA proteins or fragments, as well as combinations with other agent, are to be administered in a therapeutically effective amount to subjects in need of such treatment in the form of a pharmaceutical composition as described herein.

In certain embodiments the method comprises ameliorating, or reducing the symptoms of, or treating, or preventing disease in a subject. In certain embodiments, treatment comprises administering the anti-IL-31RA proteins, antibodies, or fragments thereof alone. In certain embodiments, treatment comprises administering the anti-IL-31RA proteins, antibodies, or fragments thereof in conjunction with a second agent used to treat, ameliorate, reduce symptoms of, or prevent the same disease or disorder or to treat a lung disease, cardiovascular disease, cancer, infectious disease, neurological disease, allergic/inflammatory disease, or metabolic disease.

Nonlimiting examples of cardiovascular diseases the antibody compositions and methods are used for ameliorating, or reducing the symptoms of, or treating, or preventing include hypertension, cardiac toxicity of anti-cancer drugs, cardiac toxicity of anthracyclines, cardiac toxicity of quinolones, heart failure regardless of origin, ischemia, heart attack, stroke, atherosclerosis, cardiac fibrillation, thrombosis and embolism.

Nonlimiting examples of infectious diseases the antibody compositions and methods are used for ameliorating, or reducing the symptoms of, or treating, or preventing include AIDS, alveolar hydatid disease (AHD, echinococcosis), amebiasis (Entamoeba histolytica infection), Angiostrongylus infection, anisakiasis, anthrax, babesiosis (Babesia infection), Balantidium infection (balantidiasis), Baylisascaris infection (raccoon roundworm), bilharzia (schistosomiasis), Blastocystis hominis infection (blastomycosis), boreliosis, botulism, Brainerd diarrhea, brucellosis, bovine spongiform encephalopathy (BSE), candidiasis, capillariasis (Capillaria infection), chronic fatigue syndrome (CFS), Chagas disease (American trypanosomiasis), chickenpox (Varicella-Zoster virus), Chlamydia pneumoniae infection, cholera, Creutzfeldt-Jakob disease (CJD), clonorchiasis (Clonorchis infection), cutaneous larva migrans (CLM) (hookworm infection), coccidioidomycosis, conjunctivitis, Coxsackievirus A16 (hand, foot and mouth disease), cryptococcosis, Cryptosporidium infection (cryptosporidiosis), Culex mosquito (West Nile virus vector), cyclosporiasis (Cyclospora infection), cysticercosis (neurocysticercosis), Cytomegalovirus infection, Dengue/Dengue fever, Dipylidium infection (dog and cat flea tapeworm), Ebola virus hemorrhagic fever, encephalitis, Entamoeba coli infection, Entamoeba dispar infection, Entamoeba hartmanni infection, Entamoeba histolytica infection (amebiasis), Entamoeba polecki infection, enterobiasis (pinworm infection), enterovirus infection (non-polio), Epstein-Barr virus infection, Escherichia coli infection, foodborne infection, foot and mouth disease, fungal dermatitis, gastroenteritis, group A streptococcal disease, group B streptococcal disease, Hansen's disease (leprosy), Hantavirus pulmonary syndrome, head lice infestation (pediculosis), Helicobacter pylori infection, hematologic disease, Hendra virus infection, hepatitis (HCV, HBV), herpes zoster (shingles), HIV Infection, human ehrlichiosis, human parainfluenza virus infection, influenza, isosporiasis (Isospora infection), Lassa fever, leishmaniasis, Kala-azar (Kala-azar, Leishmania Infection), lice (body lice, head lice, pubic lice), Lyme disease, malaria, Marburg hemorrhagic fever, measles, meningitis, mosquito-borne diseases, Mycobacterium avium complex (MAC) infection, Naegleria infection, nosocomial infections, nonpathogenic intestinal ameobae infection, onchocerciasis (river blindness), opisthorciasis (Opisthorcis infection), parvovirus infection, plague, Pneumocystis carinii pneumonia (PCP), polio, Q fever, rabies, respiratory syncytial virus (RSV) Infection, rheumatic fever, Rift Valley fever, river blindness (onchocerciasis), rotavirus infection, roundworm infection, Salmonellosis, Salmonella enteritidis, scabies, shigellosis, shingles, sleeping sickness, smallpox, streptococcal Infection, tapeworm infection (Taenia infection), tetanus, toxic shock syndrome, tuberculosis, ulcers (peptic ulcer disease), valley fever, Vibrio parahaemolyticus infection, Vibrio vulnificus infection, viral hemorrhagic fever, warts, waterborne infectious diseases, West Nile virus infection (West Nile encephalitis), whooping cough, yellow fever.

Nonlimiting examples of allergic/inflammatory conditions the antibody compositions and methods are used for ameliorating, or reducing the symptoms of, or treating, or preventing include, asthma, bronchial asthma, rheumatoid arthritis, inflammatory Bowel disease, type II diabetes, diabetes mellitus and deafness (DAD), Ballinger-Wallace syndrome, inflammatory diseases, rheumatic fever, pulmonary arterial hypertension, innate immune responses, cardiopulmonary diseases such as: chronic obstructive pulmonary disease, pulmonary embolism, pericarditis, coarctation of aorta, tetralogy of Fallot, aortic stenosis, mitral stenosis, aortic regurgitation, mitral regurgitation, pneumoconiosis, bronchiectasis, cardiomyopathies, and endothelial nitroglycerin tolerance.

Nonlimiting examples of lung diseases the antibody compositions and methods are used for ameliorating, or reducing the symptoms of, or treating, or preventing include, acute pneumonia, pulmonary fibrosis, interstitial pneumonia, pulmonary hypertension, chronic obstructive pulmonary disease (COPD), chronic bronchitis, pulmonary emphysema, asthma, refractory asthma, systemic inflammatory response syndrome (SIRS), lung injury acute (ALI), acute respiratory distress syndrome (ARDS), sarcoidosis, chronic idiopathic pulmonary thromboembolism, diffuse panbronchiolitis, cystic fibrosis, allergic alveolitis, lung cancer, obesity hypoventilation syndrome, alveolar hypoventilation syndrome and chronic transplant rejection pulmonary. Particularly important diseases are pulmonary fibrosis, interstitial pneumonia, pulmonary hypertension, asthma, COPD and SIRS.

Nonlimiting examples of cancers the antibody compositions and methods are used for ameliorating, or reducing the symptoms of, or treating, or preventing include cancers of the bladder, blood, bone, bone marrow, brain, breast, colon, esophagus, gastrointestinal tract, gum, head, kidney, liver, lung, nasopharynx, neck, ovary, prostate, skin, stomach, testis, tongue, or uterus, or malignant neoplasm, carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

Nonlimiting examples of neurological diseases the antibody compositions and methods are used for ameliorating, or reducing the symptoms of, or treating, or preventing include Alzheimer's disease, Parkinson's disease, Huntington's disease, Pick's disease, Kuf's disease, Lewy body disease, neurofibrillary tangles, Rosenthal fibers, Mallory's hyaline, senile dementia, myasthenia gravis, Gilles de la Tourette's syndrome, multiple sclerosis (MS), amyotrophic lateral sclerosis (ALS), progressive supranuclear palsy (PSP), epilepsy, Creutzfeldt-Jakob disease, deafness-dytonia syndrome, Leigh syndrome, Leber hereditary optic neuropathy (LHON), parkinsonism, dystonia, motor neuron disease, neuropathy-ataxia and retinitis pimentosa (NARP), maternal inherited Leigh syndrome (MILS), Friedreich ataxia, hereditary spastic paraplegia, Mohr-Tranebjaerg syndrome, Wilson disease, sporatic Alzheimer's disease, sporadic amyotrophic lateral sclerosis, sporadic Parkinson's disease, autonomic function disorders, hypertension, sleep disorders, neuropsychiatric disorders, depression, schizophrenia, schizoaffective disorder, korsakoffs psychosis, mania, anxiety disorders, phobic disorder, learning or memory disorders, amnesia or age-related memory loss, attention deficit disorder, dysthymic disorder, major depressive disorder, obsessive-compulsive disorder, psychoactive substance use disorders, panic disorder, bipolar affective disorder, severe bipolar affective (mood) disorder (BP-1), migraines, hyperactivity and movement disorders.

Nonlimiting examples of metabolic diseases the antibody compositions and methods are used for ameliorating, or reducing the symptoms of, or treating, or preventing include metabolic syndrome, diabetes (type 1 diabetes, type 2 diabetes, gestational diabetes, etc.), impaired glucose tolerance, obesity, diabetic neuropathy, diabetic retinopathy, diabetic nephropathy, dyslipidemia Diseases (hypertriglyceridemia, hypercholesterolemia, hypoHDLemia, postprandial hyperlipidemia, etc.), hypertension, hypertriglyceridemia, severe hypertriglyceridemia, hypercholesterolemia, familial, elevated cholesterol caused by a genetic condition, fatty liver disease, nonalcoholic fatty liver disease (NFLD), nonalcoholic steatohepatitis (NASH), dyslipidemia, mixed dyslipidemia, atherosclerosis, and coronary heart disease.

The anti-IL-31RA proteins, antibodies or antibody fragments, are optionally administered in combination with one or more active agents including other analgesic agents. Such active agents include analgesic, anti-histamine, antipyretic, anti-inflammatory, antibiotic, antiviral, and anti-cytokine agents. Active agents include agonists, antagonists, and modulators of TNF-α, IL-2, IL-4, IL-6, IL-10, IL-12, IL-13, IL-18, IFN-α, IFN-γ, BAFF, CXCL13, IP-10, VEGF, EPO, EGF, HRG, Hepatocyte Growth Factor (HGF), Hepcidin, including antibodies reactive against any of the foregoing, and antibodies reactive against any of their receptors. Active agents also include, without limitation, 2-arylpropionic acids, aceclofenac, acemetacin, acetylsalicylic acid (Aspirin), alclofenac, alminoprofen, amoxiprin, ampyrone, arylalkanoic acids, azapropazone, benorylate/benorilate, benoxaprofen, bromfenac, carprofen, celecoxib, choline magnesium salicylate, clofezone, COX-2 inhibitors, dexibuprofen, dexketoprofen, diclofenac, diflunisal, droxicam, ethenzamide, etodolac, etoricoxib, faislamine, fenamic acids, fenbufen, fenoprofen, flufenamic acid, flunoxaprofen, flurbiprofen, ibuprofen, ibuproxam, indometacin, indoprofen, kebuzone, ketoprofen, ketorolac, lomoxicam, loxoprofen, lumiracoxib, magnesium salicylate, meclofenamic acid, mefenamic acid, meloxicam, metamizole, methyl salicylate, mofebutazone, nabumetone, naproxen, n-arylanthranilic acids, nerve growth factor (NGF), oxametacin, oxaprozin, oxicams, oxyphenbutazone, parecoxib, phenazone, phenylbutazone, phenylbutazone, piroxicam, pirprofen, profens, proglumetacin, pyrazolidine derivatives, rofecoxib, salicyl salicylate, salicylamide, salicylates, sulfinpyrazone, sulindac, suprofen, tenoxicam, tiaprofenic acid, tolfenamic acid, tolmetin, and valdecoxib.

An anti-histamine can be any compound that opposes the action of histamine or its release from cells (e.g., mast cells). Anti-histamines include but are not limited to acrivastine, astemizole, azatadine, azelastine, betatastine, brompheniramine, buclizine, cetirizine, cetirizine analogues, chlorpheniramine, clemastine, CS 560, cyproheptadine, desloratadine, dexchlorpheniramine, ebastine, epinastine, fexofenadine, HSR 609, hydroxyzine, levocabastine, loratidine, methscopolamine, mizolastine, norastemizole, phenindamine, promethazine, pyrilamine, terfenadine, and tranilast.

Antibiotics include but are not limited to amikacin, aminoglycosides, amoxicillin, ampicillin, ansamycins, arsphenamine, azithromycin, azlocillin, aztreonam, bacitracin, carbacephem, carbapenems, carbenicillin, cefaclor, cefadroxil, cefalexin, cefalothin, cefalotin, cefamandole, cefazolin, cefdinir, cefditoren, cefepime, cefixime, cefoperazone, cefotaxime, cefoxitin, cefpodoxime, cefprozil, ceftazidime, ceftibuten, ceftizoxime, ceftobiprole, ceftriaxone, cefuroxime, cephalosporins, chloramphenicol, cilastatin, ciprofloxacin, clarithromycin, clindamycin, cloxacillin, colistin, co-trimoxazole, dalfopristin, demeclocycline, dicloxacillin, dirithromycin, doripenem, doxycycline, enoxacin, ertapenem, erythromycin, ethambutol, flucloxacillin, fosfomycin, furazolidone, fusidic acid, gatifloxacin, geldanamycin, gentamicin, glycopeptides, herbimycin, imipenem, isoniazid, kanamycin, levofloxacin, lincomycin, linezolid, lomefloxacin, loracarbef, macrolides, mafenide, meropenem, meticillin, metronidazole, mezlocillin, minocycline, monobactams, moxifloxacin, mupirocin, nafcillin, neomycin, netilmicin, nitrofurantoin, norfloxacin, ofloxacin, oxacillin, oxytetracycline, paromomycin, penicillin, penicillins, piperacillin, platensimycin, polymyxin B, polypeptides, prontosil, pyrazinamide, quinolones, quinupristin, rifampicin, rifampin, roxithromycin, spectinomycin, streptomycin, sulfacetamide, sulfamethizole, sulfanilimide, sulfasalazine, sulfisoxazole, sulfonamides, teicoplanin, telithromycin, tetracycline, tetracyclines, ticarcillin, tinidazole, tobramycin, trimethoprim, trimethoprim-sulfamethoxazole, troleandomycin, trovafloxacin, and vancomycin.

Active agents also include aldosterone, beclometasone, betamethasone, corticosteroids, cortisol, cortisone acetate, deoxycorticosterone acetate, dexamethasone, fludrocortisone acetate, glucocorticoids, hydrocortisone, methylprednisolone, prednisolone, prednisone, steroids, and triamcinolone. Any suitable combination of these active agents is also contemplated.

### Formulations and Methods of Administration

For in vivo use, a therapeutic agent as described herein is generally incorporated into a pharmaceutical composition prior to administration. Within such compositions, one or more therapeutic compounds as described herein are present as active ingredient(s) (i.e., are present at levels sufficient to provide a statistically significant effect on the symptoms of cystic fibrosis, as measured using a representative assay). A pharmaceutical composition comprises one or more such compounds in combination with any pharmaceutically acceptable carrier(s) known to those skilled in the art to be suitable for the particular mode of administration. In addition, other pharmaceutically active ingredients (including other therapeutic agents) may, but need not, be present within the composition.

The antibodies of the present invention can be formulated according to standard methods (see, for example, Remington's Pharmaceutical Science, latest edition, Mark Publishing Company, Easton, U.S.A.), and may comprise pharmaceutically acceptable carriers and/or additives. The present invention relates to compositions (including reagents and pharmaceuticals) comprising the antibodies of the invention, and pharmaceutically acceptable carriers and/or additives. Exemplary carriers include surfactants (for example, PEG and Tween), excipients, antioxidants (for example, ascorbic acid), coloring agents, flavoring agents, preservatives, stabilizers, buffering agents (for example, phosphoric acid, citric acid, and other organic acids), chelating agents (for example, EDTA), suspending agents, isotonizing agents, binders, disintegrators, lubricants, fluidity promoters, and corrigents. However, the carriers that may be employed in the present invention are not limited to this list. In fact, other commonly used carriers can be appropriately employed: light anhydrous silicic acid, lactose, crystalline cellulose, mannitol, starch, carmelose calcium, carmelose sodium, hydroxypropylcellulose, hydroxypropylmethyl cellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, inorganic salt, and so on. The composition may also comprise other low-molecular-weight polypeptides, proteins such as serum albumin, gelatin, and immunoglobulin, and amino acids such as glycine, glutamine, asparagine, arginine, and lysine. When the composition is prepared as an aqueous solution for injection, it can comprise an isotonic solution comprising, for example, physiological saline, dextrose, and other adjuvants, including, for example, D-sorbitol, D-mannose, D-mannitol, and sodium chloride, which can also contain an appropriate solubilizing agent, for example, alcohol (for example, ethanol), polyalcohol (for example, propylene glycol and PEG), and non-ionic detergent (polysorbate 80 and HCO-50).

If necessary, antibodies of the present invention may be encapsulated in microcapsules (microcapsules made of hydroxycellulose, gelatin, polymethylmethacrylate, and the like), and made into components of colloidal drug delivery systems (liposomes, albumin microspheres, microemulsions, nano-particles, and nano-capsules) (for example, see “Remington's Pharmaceutical Science 16th edition”, Oslo Ed. (1980)). Moreover, methods for making sustained-release drugs are known, and these can be applied for the antibodies of the present invention (Langer et al., J. Biomed. Mater. Res. 15: 167-277 (1981); Langer, Chem. Tech. 12: 98-105 (1982); U.S. Pat. No. 3,773,919; EP Patent Application No. 58,481; Sidman et al., Biopolymers 22: 547-556 (1983); EP: 133,988).

The term “therapeutically effective amount,” in reference to treating a disease state/condition, refers to an amount of a compound either alone or as contained in a pharmaceutical composition that is capable of having any detectable, positive effect on any symptom, aspect, or characteristics of a disease state/condition when administered as a single dose or in multiple doses. Such effect need not be absolute to be beneficial.

The terms “treat,” “treating” and “treatment” as used herein include administering a compound prior to the onset of clinical symptoms of a disease state/condition so as to prevent any symptom, as well as administering a compound after the onset of clinical symptoms of a disease state/condition so as to reduce or eliminate any symptom, aspect or characteristic of the disease state/condition. Such treating need not be absolute to be useful.

In certain embodiments, the present therapeutic agent may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Useful dosages of the compounds of the present invention can be determined by comparing their in vitro activity, and in vivo activity in animal models. In certain embodiments, a useful dose is from about 0.1 mg/kg to about 5 mg/kg or from about 0.5 mg/kg to about 2 mg/kg. Methods for the extrapolation of effective dosages in humans and animals of different sizes are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently administered in unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 0.5 to about 75 μM, preferably, about 1 to 50 μM, most preferably, about 2 to about 30 M. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

Exemplary IL-31Rα receptor constructs useful for screening, identifying, and evaluating anti-IL-31Rα antibodies that block receptor binding include the following:

i) Signal peptide-amino acids 20-518 of extracellular domain (ECD) of
canine IL-31Rα-2XGGGS (SEQ ID NO: 81) linker-murine IgG2A Fc
(SEQ ID NO: 5)
MGWSCIILFLVATATGVHSVLPAKPENISCIFYYEENFTCTWSPEKEASYTWYKVKRTYS
YGYKSDICSTDNSTRGNHASCSFLPPTITNPDNYTIQVEAQNADGIMKSDITYWNLDAIM
KIEPPEIFSVKSVLGIKRMLQIKWIRPVLAPHSSTLKYTLRFRTINSAYWMEVNFTKEDID
RDETYNLTELQAFTEYVMTLRCAPAESMFWSGWSQEKVGTTEEEAPYGLDLWRVLKP
AMVDGRRPVQLMWKKATGAPVLEKALGYNIWYFPENNTNLTETVNTTNQTHELYLG
GKTYWVYVVSYNSLGESPVATLRIPALNEKTFQCIEAMQACLTQDQLVVEWQSSAPEV
DTWMVEWFPDVDSEPSSFSWESVSQARNWTIQKDELKPLWCYNISVYPVLRDRVGQPY
STQAYVQEGIPSAGPVTQADSIGVKTVTITWKEIPKSKRNGFIKNYTIFYQAEDGKEFSKT
VNSNILQYRLESLTRRTSYSLQVMASTNAGGTNGTKINFKTLSISVLGGGSGGGSEPRGP
TIKPCPPCKCPAPNLLGGPSVFIFPPKIKDVLMISLSPIVTCVVVDVSEDDPDVQISWFVNN
VEVHTAQTQTHREDYNSTLRVVSALPIQHQDWMSGKEFKCKVNNKDLPAPIERTISKPK
GSVRAPQVYVLPPPEEEMTKKQVTLTCMVTDFMPEDIYVEWTNNGKTELNYKNTEPVL
DSDGSYFMYSKLRVEKKNWVERNSYSCSVVHEGLHNHHTTKSFSRTPG*
ii) Signal peptide-amino acids 20-308 of extracellular domain (ECD) of
canine IL-31Rα-AviTag-His Tag
(SEQ ID NO: 28)
MGWSCIILFLVATATGVHSVLPAKPENISCIFYYEENFTCTWSPEKEASYTWYKVKRTYS
YGYKSDICSTDNSTRGNHASCSFLPPTITNPDNYTIQVEAQNADGIMKSDITYWNLDAIM
KIEPPEIFSVKSVLGIKRMLQIKWIRPVLAPHSSTLKYTLRFRTINSAYWMEVNFTKEDID
RDETYNLTELQAFTEYVMTLRCAPAESMFWSGWSQEKVGTTEEEAPYGLDLWRVLKP
AMVDGRRPVQLMWKKATGAPVLEKALGYNIWYFPENNTNLTETVNTTNQTHELYLG
GKTYWVYVVSYNSLGESPVATLRIPALNEKTGLNDIFEAQKIEWHEHHHHHHHH*

Without limitation, and subject to sequence variation disclosed herein, binding proteins of the invention may comprise one or more CDRs or variable domains of the following heavy and light chains (Table 1). Boundaries between variable and constant domains are depicted. Signal peptides are shown at the amino terminus for SEQ ID NO:6-13.

TABLE 1
Heavy and Light Chain Sequences

| SEQ | | |
|---|---|---|
| ID | | |
| NO: | Clone | Sequence |
| 1 | Clone 109 | EVQLVESGGG VVRPGGSLRL SCAASGFTFD DYGMSWVRQI PGRGLEWVSG |
| | VH | INWAGGTIGY ADSVKGRFTV SRDDANNSLY LQMNSLRAED TALYLCARES |
| | | PLRLGDLGGD YFYYYYYMDV WGKGTTVTVS S |
| | | ASTKGPSVF PLAPSSKSTS |
| | | GGTAALGCLV KDYFPEPVTV SWNSGALTSG VHTFPAVLQS SGLYSLSSVV |
| | | TVPSSSLGTQ TYICNVNHKP SNTKVDKKVE PKSCDKTHTC PPCPAPEAAG |
| | | GPSVFLFPPK PKDTLMISRT PEVTCVVVDV SHEDPEVKFN WYVDGVEVHN |
| | | AKTKPREEQY NSTYRVVSVL TVLHQDWLNG KEYKCKVSNK ALPAPIEKTI |
| | | SKAKGQPREP QVYTLPPSRE EMTKNQVSLT CLVKGFYPSD IAVEWESNGQ |
| | | PENNYKTTPP VLDSDGSFFL YSKLTVDKSR WQQGNVFSCS VMHEALHNHY |
| | | TQKSLSLSPG |
| 2 | Clone 109 | EIVLTQSPGT LSLSPGERAT LSCRASQSVS SRYLVWYQQK PGQAPRLLIY |
| | VL | GTSSRATGIP DRFSGSGSGT DFTLTISRLE PEDFAVYYCQ QYDNSPRTFG |
| | | QGTKVEIK |
| | | RT VAAPSVFIFP PSDEQLKSGT ASVVCLLNNF YPREAKVQWK |
| | | VDNALQSGNS QESVTEQDSK DSTYSLSSTL TLSKADYEKH KVYACEVTHQ |
| | | GLSSPVTKSF NRGEC |
| 3 | Clone 186 | EVQLLESGGG LIQPGGSLRL SCGASGFTFS SYAMSWVRQA PGKGLEWVSA |
| | VH | ISGSGNSTYY ADSVKGRFTI SRDNSKNTLY LQMNSLRAED TAVYYCATQV |
| | | VYYFKMDVWG KGTTVTVSS |
| | | A STKGPSVFPL APSSKSTSGG TAALGCLVKD |
| | | YFPEPVTVSW NSGALTSGVH TFPAVLQSSG LYSLSSVVTV PSSSLGTQTY |
| | | ICNVNHKPSN TKVDKKVEPK SCDKTHTCPP CPAPEAAGGP SVFLFPPKPK |
| | | DTLMISRTPE VTCVVVDVSH EDPEVKFNWY VDGVEVHNAK TKPREEQYNS |
| | | TYRVVSVLTV LHQDWLNGKE YKCKVSNKAL PAPIEKTISK AKGQPREPQV |
| | | YTLPPSREEM TKNQVSLTCL VKGFYPSDIA VEWESNGQPE NNYKTTPPVL |
| | | DSDGSFFLYS KLTVDKSRWQ QGNVFSCSVM HEALHNHYTQ KSLSLSPG* |
| 4 | Clone 186 | EIVMTQSPAT LSVSPGERAT LSCRASQSVS SNLAWYRQKP GQAPRLLIYG |
| | VL | ASTRATGIPA RFSGSGSGTE FTLTISSLQS EDFAVYYCQQ YYNWPPFTFG |
| | | PGTKLDIK |
| | | RT VAAPSVFIFP PSDEQLKSGT ASVVCLLNNF YPREAKVQWK |
| | | VDNALQSGNS QESVTEQDSK DSTYSLSSTL TLSKADYEKH KVYACEVTHQ |
| | | GLSSPVTKSF NRGEC* |
| 6 | Clone 109 | MGWSCIILFL VATATGVHS |
| | canine | EVQLVESGGG VVRPGGSLRL SCAASGFTFD DYGMSWVRQI PGRGLEWVSG |
| | chimeric | INWAGGTIGY ADSVKGRFTV SRDDANNSLY LQMNSLRAED TALYLCARES |
| | clone H | PLRLGDLGGD YFYYYYYMDV WGKGTTVTVS S |
| | chain | ASTTAPSVF PLAPSCGSTS |
| | | GSTVALACLV SGYFPEPVTV SWNSGSLTSG VHTFPSVLQS SGLYSLSSMV |
| | | TVPSSRWPSE TFTCNVAHPA SKTKVDKPVP KRENGRVPRP PDCPKCPAPE |
| | | AAGGPSVFIF PPKPKDTLLI ARTPEVTCVV VDLDPEDPEV QISWFVDGKQ |
| | | MQLAKTQPRE EQFNGTYRVV SVLPIGHQDW LKGKQFTCKV NNKALPSPIE |
| | | RTISKARGQA HQPSVYVLPP SREELSKNTV SLTCLIKDFF PPDIDVEWQS |
| | | NGQQEPESKY RTTPPQLDED GSYFLYSKLS VDKSRWQRGD TFICYVMHEA |
| | | LHNHYTQESL SHSPG* |
| 7 | Clone 109 | METDTLLLWV LLLWVPGSTG |
| | canine | EIVLTQSPGT LSLSPGERAT LSCRASQSVS SRYLVWYQQK PGQAPRLLIY |
| | chimeric | GTSSRATGIP DRFSGSGSGT DFTLTISRLE PEDFAVYYCQ QYDNSPRTFG |
| | clone L | QGTKVEIK |
| | chain | RN DAQPAVYLFQ PSPDQLHTGS ASVVCLLNSF YPKDINVKWK |
| | | VDGVIQDTGI QESVTEQDKD STYSLSSTLT MSSTEYLSHE LYSCEITHKS |
| | | LPSTLIKSFQ RSECQRVD* |
| 8 | Clone 186 | MGWSCIILFL VATATGVHS |
| | canine | EVQLLESGGG LIQPGGSLRL SCGASGFTFS SYAMSWVRQA PGKGLEWVSA |
| | chimeric | ISGSGNSTYY ADSVKGRFTI SRDNSKNTLY LQMNSLRAED TAVYYCATQV |
| | clone H | VYYFKMDVWG KGTTVTVSS |
| | chain | A STTAPSVFPL APSCGSTSGS TVALACLVSG |
| | | YFPEPVTVSW NSGSLTSGVH TFPSVLQSSG LYSLSSMVTV PSSRWPSETF |
| | | TCNVAHPASK TKVDKPVPKR ENGRVPRPPD CPKCPAPEAA GGPSVFIFPP |
| | | KPKDTLLIAR TPEVTCVVVD LDPEDPEVQI SWFVDGKQMQ LAKTQPREEQ |
| | | FNGTYRVVSV LPIGHQDWLK GKQFTCKVNN KALPSPIERT ISKARGQAHQ |
| | | PSVYVLPPSR EELSKNTVSL TCLIKDFFPP DIDVEWQSNG QQEPESKYRT |
| | | TPPQLDEDGS YFLYSKLSVD KSRWQRGDTF ICYVMHEALH NHYTQESLSH |
| | | SPG* |
| 9 | Clone 186 | METDTLLLWV LLLWVPGSTG |
| | canine | EIVMTQSPAT LSVSPGERAT LSCRASQSVS SNLAWYRQKP GQAPRLLIYG |
| | chimeric | ASTRATGIPA RFSGSGSGTE FTLTISSLQS EDFAVYYCQQ YYNWPPFTFG |
| | clone L | PGTKLDIK |
| | chain | RN DAQPAVYLFQ PSPDQLHTGS ASVVCLLNSF YPKDINVKWK |
| | | VDGVIQDTGI QESVTEQDKD STYSLSSTLT MSSTEYLSHE LYSCEITHKS |
| | | LPSTLIKSFQ RSECQRVD* |
| 10 | Clone | MGWSCIILFL VATATGVHS |
| | 109-49 H | EVQLVESGGD LVKPGGSLRL SCVASGFTFD DYGMSWVRQA PGKGLQWVSG |
| | chain | INWAGGTIGY ADAVKGRFTI SRDNARNTVY LQMNSLRAED TAVYYCARES |
| | | PLRLGDLGGD YFYYYYYMDV WGQGTLVTVS S |
| | | ASTTAPSVE PLAPSCGSTS |
| | | GSTVALACLV SGYFPEPVTV SWNSGSLTSG VHTFPSVLQS SGLYSLSSMV |
| | | TVPSSRWPSE TFTCNVAHPA SKTKVDKPVP KRENGRVPRP PDCPKCPAPE |
| | | AAGGPSVFIF PPKPKDTLLI ARTPEVTCVV VDLDPEDPEV QISWFVDGKQ |
| | | MQLAKTQPRE EQFNGTYRVV SVLPIGHQDW LKGKQFTCKV NNKALPSPIE |
| | | RTISKARGQA HQPSVYVLPP SREELSKNTV SLTCLIKDFF PPDIDVEWQS |
| | | NGQQEPESKY RTTPPQLDED GSYFLYSKLS VDKSRWQRGD TFICYVMHEA |
| | | LHNHYTQESL SHSPG* |
| 11 | Clone | METDTLLLWV LLLWVPGSTG |
| | 109-49 L | EVVMIQTPLS LSVSPGEPAS ISCRASQSVS SRYLVWYLQK PGQSPQLLIY |
| | chain | GTSSRATGVS DRFSGSGSGT DFTLRISRVE AEDVGVYYCQ QYDNSPRTFG |
| | | QGTKVEIK |
| | | RN DAQPAVYLFQ PSPDQLHTGS ASVVCLLNSF YPKDINVKWK |
| | | VDGVIQDTGI QESVTEQDKD STYSLSSTLT MSSTEYLSHE LYSCEITHKS |
| | | LPSTLIKSFQ RSECQRVD* |
| 12 | Clone | MGWSCIILFL VATATGVHS |
| | 186-95 H | EVQLVESGGD LVKPGGSLRL SCVASGFTFS SYAMSWVRQA PGKGLQWVSA |
| | chain | ISGSGNSTYY ADAVKGRFTI SRDNAKNTLY LQMNSLRAED TAVYYCATQV |
| | | VYYFKMDVWG QGTLVTVSS |
| | | A STTAPSVFPL APSCGSTSGS TVALACLVSG |
| | | YFPEPVTVSW NSGSLTSGVH TFPSVLQSSG LYSLSSMVTV PSSRWPSETF |
| | | TCNVAHPASK TKVDKPVPKR ENGRVPRPPD CPKCPAPEAA GGPSVFIFPP |
| | | KPKDTLLIAR TPEVTCVVVD LDPEDPEVQI SWFVDGKQMQ LAKTQPREEQ |
| | | FNGTYRVVSV LPIGHQDWLK GKQFTCKVNN KALPSPIERT ISKARGQAHQ |
| | | PSVYVLPPSR EELSKNTVSL TCLIKDFFPP DIDVEWQSNG QQEPESKYRT |
| | | TPPQLDEDGS YFLYSKLSVD KSRWQRGDTF ICYVMHEALH NHYTQESLSH |
| | | SPG* |
| 13 | Clone | METDTLLLWV LLLWVPGSTG |
| | 186-95 L | EIVMTQSPGS LAGSAGESVS INCKSSQSVS SNLAWYQQKP GERPKLLIYG |
| | chain | ASTRASGVPA RFSSSGSGTD FTLTINNLQA EDVGDYYCQQ YYNWPPFTFG |
| | | QGTKLEIK |
| | | RN DAQPAVYLFQ PSPDQLHTGS ASVVCLLNSF YPKDINVKWK |
| | | VDGVIQDTGI QESVTEQDKD STYSLSSTLT MSSTEYLSHE LYSCEITHKS |
| | | LPSTLIKSFQ RSECQRVD* |

Without limitation, and subject to sequence variation disclosed herein, binding proteins of the invention may comprise one or more CDRs or variable domains of the following of the following heavy and light chains (Table 2). Boundaries of variable and constant domains are depicted. Signal peptides are shown at the amino terminus for SEQ ID NO:14-20.

TABLE 2
Clone 109-49 Heavy and Light Chain Sequence Variants

| | | |
|---|---|---|
| 14 | Clone 109- | MGWSCIILFL VATATGVHS |
| | 49 Heavy | EVQLVESGGD LVKPGGSLRL SCVASGFTED DYGMSWVRQA PGKGLQWVSG |
| | Chain W53Y | INYAGGTIGY ADAVKGRFTI SRDNARNTVY LQMNSLRAED TAVYYCARES |
| | variant | PLRLGDLGGD YFYYYYYMDV WGQGTLVTVS S |
| | | ASTTAPSVF PLAPSCGSTS |
| | | GSTVALACLV SGYFPEPVTV SWNSGSLTSG VHTFPSVLQS SGLYSLSSMV |
| | | TVPSSRWPSE TFTCNVAHPA SKTKVDKPVP KRENGRVPRP PDCPKCPAPE |
| | | AAGGPSVFIF PPKPKDTLLI ARTPEVTCVV VDLDPEDPEV QISWFVDGKQ |
| | | MQLAKTQPRE EQFNGTYRVV SVLPIGHQDW LKGKQFTCKV NNKALPSPIE |
| | | RTISKARGQA HQPSVYVLPP SREELSKNTV SLTCLIKDEF PPDIDVEWQS |
| | | NGQQEPESKY RTTPPQLDED GSYFLYSKLS VDKSRWQRGD TFICYVMHEA |
| | | LHNHYTQESL SHSPG* |
| 15 | Clone 109- | MGWSCIILFL VATATGVHS |
| | 49 Heavy | EVQLVESGGD LVKPGGSLRL SCVASGFTFD DYGMSWVRQA PGKGLQWVSG |
| | Chain W53F | INFAGGTIGY ADAVKGRFTI SRDNARNTVY LQMNSLRAED TAVYYCARES |
| | variant | PLRLGDLGGD YFYYYYYMDV WGQGTLVTVS S |
| | | ASTTAPSVF PLAPSCGSTS |
| | | GSTVALACLV SGYFPEPVTV SWNSGSLTSG VHTFPSVLQS SGLYSLSSMV |
| | | TVPSSRWPSE TFTCNVAHPA SKTKVDKPVP KRENGRVPRP PDCPKCPAPE |
| | | AAGGPSVFIF PPKPKDTLLI ARTPEVTCVV VDLDPEDPEV QISWFVDGKQ |
| | | MQLAKTQPRE EQFNGTYRVV SVLPIGHQDW LKGKQFTCKV NNKALPSPIE |
| | | RTISKARGQA HQPSVYVLPP SREELSKNTV SLTCLIKDFF PPDIDVEWQS |
| | | NGQQEPESKY RTTPPQLDED GSYFLYSKLS VDKSRWQRGD TFICYVMHEA |
| | | LHNHYTQESL SHSPG* |
| 16 | Clone 109- | MGWSCIILFL VATATGVHS |
| | 49 Heavy | EVQLVESGGD LVKPGGSLRL SCVASGFTFD DYGMSWVRQA PGKGLQWVSG |
| | Chain M118L | INWAGGTIGY ADAVKGRFTI SRDNARNTVY LQMNSLRAED TAVYYCARES |
| | variant | PLRLGDLGGD YFYYYYYLDV WGQGTLVTVS S |
| | | ASTTAPSVF PLAPSCGSTS |
| | | GSTVALACLV SGYFPEPVTV SWNSGSLTSG VHTFPSVLQS SGLYSLSSMV |
| | | TVPSSRWPSE TFTCNVAHPA SKTKVDKPVP KRENGRVPRP PDCPKCPAPE |
| | | AAGGPSVFIF PPKPKDTLLI ARTPEVTCVV VDLDPEDPEV QISWFVDGKQ |
| | | MQLAKTQPRE EQFNGTYRVV SVLPIGHQDW LKGKQFTCKV NNKALPSPIE |
| | | RTISKARGQA HQPSVYVLPP SREELSKNTV SLTCLIKDFF PPDIDVEWQS |
| | | NGQQEPESKY RTTPPQLDED GSYFLYSKLS VDKSRWQRGD TFICYVMHEA |
| | | LHNHYTQESL SHSPG* |
| 17 | Clone 109- | MGWSCIILFL VATATGVHS |
| | 49 Heavy | EVQLVESGGD LVKPGGSLRL SCVASGFTFD DYGMSWVRQA PGKGLQWVSG |
| | Chain M118I | INWAGGTIGY ADAVKGRFTI SRDNARNTVY LQMNSLRAED TAVYYCARES |
| | variant | PLRLGDLGGD YFYYYYYIDV WGQGTLVTVS S |
| | | ASTTAPSVF PLAPSCGSTS |
| | | GSTVALACLV SGYFPEPVTV SWNSGSLTSG VHTFPSVLQS SGLYSLSSMV |
| | | TVPSSRWPSE TFTCNVAHPA SKTKVDKPVP KRENGRVPRP PDCPKCPAPE |
| | | AAGGPSVFIF PPKPKDTLLI ARTPEVTCVV VDLDPEDPEV QISWFVDGKQ |
| | | MQLAKTQPRE EQFNGTYRVV SVLPIGHQDW LKGKQFTCKV NNKALPSPIE |
| | | RTISKARGQA HQPSVYVLPP SREELSKNTV SLTCLIKDFF PPDIDVEWQS |
| | | NGQQEPESKY RTTPPQLDED GSYFLYSKLS VDKSRWQRGD TFICYVMHEA |
| | | LHNHYTQESL SHSPG* |
| 18 | Clone 109- | MGWSCIILFL VATATGVHS |
| | 49 Heavy | EVQLVESGGD LVKPGGSLRL SCVASGFTFD DYGMSWVRQA PGKGLQWVSG |
| | Chain | INFAGGTIGY ADAVKGRFTI SRDNARNTVY LQMNSLRAED TAVYYCARES |
| | W53F + | PLRLGDLGGD YFYYYYYIDV WGQGTLVTVS S |
| | M118I | ASTTAPSVF PLAPSCGSTS |
| | variant | GSTVALACLV SGYFPEPVTV SWNSGSLTSG VHTFPSVLQS SGLYSLSSMV |
| | | TVPSSRWPSE TFTCNVAHPA SKTKVDKPVP KRENGRVPRP PDCPKCPAPE |
| | | AAGGPSVFIF PPKPKDTLLI ARTPEVTCVV VDLDPEDPEV QISWFVDGKQ |
| | | MQLAKTQPRE EQFNGTYRVV SVLPIGHQDW LKGKQFTCKV NNKALPSPIE |
| | | RTISKARGQA HQPSVYVLPP SREELSKNTV SLTCLIKDFF PPDIDVEWQS |
| | | NGQQEPESKY RTTPPQLDED GSYFLYSKLS VDKSRWQRGD TFICYVMHEA |
| | | LHNHYTQESL SHSPG* |
| 19 | Clone 109- | METDTLLLWV LLLWVPGSTG |
| | 49 Light | EVVMIQTPLS LSVSPGEPAS ISCRASQSVS SRYLVWYLQK PGQSPQLLIY |
| | chain N94Q | GTSSRATGVS DRFSGSGSGT DFTLRISRVE AEDVGVYYCQ QYDQSPRTFG |
| | variant | QGTKVEIK |
| | | RN DAQPAVYLFQ PSPDQLHTGS ASVVCLLNSF YPKDINVKWK |
| | | VDGVIQDTGI QESVTEQDKD STYSLSSTLT MSSTEYLSHE LYSCEITHKS |
| | | LPSTLIKSFQ RSECQRVD* |
| 20 | Clone 109- | METDTLLLWVLLLWVPGSTG |
| | 49 Light | EVVMIQTPLS LSVSPGEPAS ISCRASQSVS SRYLVWYLQK PGQSPQLLIY |
| | chain S95A | GTSSRATGVS DRFSGSGSGT DFTLRISRVE AEDVGVYYCQ QYDNAPRTFG |
| | variant | QGTKVEIK |
| | | RN DAQPAVYLFQ PSPDQLHTGS ASVVCLLNSF YPKDINVKWK |
| | | VDGVIQDTGI QESVTEQDKD STYSLSSTLT MSSTEYLSHE LYSCEITHKS |
| | | LPSTLIKSFQ RSECQRVD* |

Without limitation, and subject to sequence variation disclosed herein, binding proteins of the invention may comprise one or more CDRs or variable domains of the following of the following heavy and light chains (Table 3). Boundaries of variable and constant domains are depicted. Signal peptides are shown at the amino terminus for SEQ ID NO:21-27.

TABLE 3
Clone 186-95 Heavy and Light Chain Sequence Variants

| | | |
|---|---|---|
| 21 | Clone 186- | MGWSCIILFLVATATGVHS |
| | 95 heavy | EVQLVESGGD LVKPGGSLRL SCVASGFTFS SYAMSWVRQA PGKGLQWVSA |
| | chain N56Q | ISGSGQSTYY ADAVKGRFTI SRDNAKNTLY LQMNSLRAED TAVYYCATQV |
| | variant | VYYFKMDVWG QGTLVTVSS |
| | | A STTAPSVFPL APSCGSTSGS TVALACLVSG |
| | | YFPEPVTVSW NSGSLTSGVH TFPSVLQSSG LYSLSSMVTV PSSRWPSETF |
| | | TCNVAHPASK TKVDKPVPKR ENGRVPRPPD CPKCPAPEAA GGPSVFIFPP |
| | | KPKDTLLIAR TPEVTCVVVD LDPEDPEVQI SWFVDGKQMQ LAKTQPREEQ |
| | | FNGTYRVVSV LPIGHQDWLK GKQFTCKVNN KALPSPIERT ISKARGQAHQ |
| | | PSVYVLPPSR EELSKNTVSL TCLIKDFFPP DIDVEWQSNG QQEPESKYRT |
| | | TPPQLDEDGS YFLYSKLSVD KSRWQRGDTF ICYVMHEALH NHYTQESLSH |
| | | SPG* |
| 22 | Clone 186- | MGWSCIILFL VATATGVHS |
| | 95 heavy | EVQLVESGGD LVKPGGSLRL SCVASGFTFS SYAMSWVRQA PGKGLQWVSA |
| | chain S57A | ISGSGNATYY ADAVKGRFTI SRDNAKNTLY LQMNSLRAED TAVYYCATQV |
| | variant | VYYFKMDVWG QGTLVTVSS |
| | | A STTAPSVFPL APSCGSTSGS TVALACLVSG |
| | | YFPEPVTVSW NSGSLTSGVH TFPSVLQSSG LYSLSSMVTV PSSRWPSETF |
| | | TCNVAHPASK TKVDKPVPKR ENGRVPRPPD CPKCPAPEAA GGPSVFIFPP |
| | | KPKDTLLIAR TPEVTCVVVD LDPEDPEVQI SWFVDGKQMQ LAKTQPREEQ |
| | | FNGTYRVVSV LPIGHQDWLK GKQFTCKVNN KALPSPIERT ISKARGQAHQ |
| | | PSVYVLPPSR EELSKNTVSL TCLIKDFFPP DIDVEWQSNG QQEPESKYRT |
| | | TPPQLDEDGS YFLYSKLSVD KSRWQRGDTF ICYVMHEALH NHYTQESLSH |
| | | SPG* |
| 23 | Clone 186- | MGWSCIILFL VATATGVHS |
| | 95 heavy | EVQLVESGGD LVKPGGSLRL SCVASGFTFS SYAMSWVRQA PGKGLQWVSA |
| | chain | ISGSGNSTYY ADAVKGRFTI SRDNAKNTLY LQMNSLRAED TAVYYCATQV |
| | M106L | VYYFKLDVWG QGTLVTVSS |
| | variant | A STTAPSVFPL APSCGSTSGS TVALACLVSG |
| | | YFPEPVTVSW NSGSLTSGVH TEPSVLQSSG LYSLSSMVTV PSSRWPSETF |
| | | TCNVAHPASK TKVDKPVPKR ENGRVPRPPD CPKCPAPEAA GGPSVFIFPP |
| | | KPKDTLLIAR TPEVTCVVVD LDPEDPEVQI SWFVDGKQMQ LAKTQPREEQ |
| | | FNGTYRVVSV LPIGHQDWLK GKQFTCKVNN KALPSPIERT ISKARGQAHQ |
| | | PSVYVLPPSR EELSKNTVSL TCLIKDFFPP DIDVEWQSNG QQEPESKYRT |
| | | TPPQLDEDGS YFLYSKLSVD KSRWQRGDTE ICYVMHEALH NHYTQESLSH |
| | | SPG* |
| 24 | Clone 186- | MGWSCIILFL VATATGVHS |
| | 95 heavy | EVQLVESGGD LVKPGGSLRL SCVASGFTFS SYAMSWVRQA PGKGLQWVSA |
| | chain | ISGSGNSTYY ADAVKGRFTI SRDNAKNTLY LQMNSLRAED TAVYYCATQV |
| | M106I | VYYFKIDVWG QGTLVTVSS |
| | variant | A STTAPSVFPL APSCGSTSGS TVALACLVSG |
| | | YFPEPVTVSW NSGSLTSGVH TFPSVLQSSG LYSLSSMVTV PSSRWPSETF |
| | | TCNVAHPASK TKVDKPVPKR ENGRVPRPPD CPKCPAPEAA GGPSVFIFPP |
| | | KPKDTLLIAR TPEVTCVVVD LDPEDPEVQI SWFVDGKQMQ LAKTQPREEQ |
| | | FNGTYRVVSV LPIGHQDWLK GKQFTCKVNN KALPSPIERT ISKARGQAHQ |
| | | PSVYVLPPSR EELSKNTVSL TCLIKDFFPP DIDVEWQSNG QQEPESKYRT |
| | | TPPQLDEDGS YFLYSKLSVD KSRWQRGDTF ICYVMHEALH NHYTQESLSH |
| | | SPG* |
| 25 | Clone 186- | MGWSCIILFLVATATGVHS |
| | 95 heavy | EVQLVESGGD LVKPGGSLRL SCVASGFTFS SYAMSWVRQA PGKGLQWVSA |
| | chain N56Q | ISGSGQSTYY ADAVKGRFTI SRDNAKNTLY LQMNSLRAED TAVYYCATQV |
| | + M106L | VYYFKLDVWG QGTLVTVSS |
| | variant | A STTAPSVFPL APSCGSTSGS TVALACLVSG |
| | | YFPEPVTVSW NSGSLTSGVH TFPSVLQSSG LYSLSSMVTV PSSRWPSETF |
| | | TCNVAHPASK TKVDKPVPKR ENGRVPRPPD CPKCPAPEAA GGPSVFIFPP |
| | | KPKDTLLIAR TPEVTCVVVD LDPEDPEVQI SWFVDGKQMQ LAKTQPREEQ |
| | | FNGTYRVVSV LPIGHQDWLK GKQFTCKVNN KALPSPIERT ISKARGQAHQ |
| | | PSVYVLPPSR EELSKNTVSL TCLIKDFFPP DIDVEWQSNG QQEPESKYRT |
| | | TPPQLDEDGS YFLYSKLSVD KSRWQRGDTF ICYVMHEALH NHYTQESLSH |
| | | SPG* |
| 26 | Clone 186- | METDTLLLWVLLLWVPGSTG |
| | 95 light | EIVMTQSPGS LAGSAGESVS INCKSSQSVS SNLAWYQQKP GERPKLLIYG |
| | chain W94Y | ASTRASGVPA RFSSSGSGTD FTLTINNLQA EDVGDYYCQQ YYNYPPFTFG |
| | variant | QGTKLEIK |
| | | RN DAQPAVYLFQ PSPDQLHTGS ASVVCLLNSF YPKDINVKWK |
| | | VDGVIQDTGI QESVTEQDKD STYSLSSTLT MSSTEYLSHE LYSCEITHKS |
| | | LPSTLIKSFQ RSECQRVD* |
| 27 | Clone 186- | METDTLLLWVLLLWVPGSTG |
| | 95 light | EIVMTQSPGS LAGSAGESVS INCKSSQSVS SNLAWYQQKP GERPKLLIYG |
| | chain W94F | ASTRASGVPA RFSSSGSGTD FTLTINNLQA EDVGDYYCQQ YYNFPPFTFG |
| | variant | QGTKLEIK |
| | | RN DAQPAVYLFQ PSPDQLHTGS ASVVCLLNSF YPKDINVKWK |
| | | VDGVIQDTGI QESVTEQDKD STYSLSSTLT MSSTEYLSHE LYSCEITHKS |
| | | LPSTLIKSFQ RSECQRVD* |

Although the present invention and its advantages have been described in detail, it should be understood that various changes, substitutions and alterations can be made herein without departing from the spirit and scope of the invention as defined in the appended claims. The present invention will be further illustrated in the following Examples which are given for illustration purposes only and are not intended to limit the invention in any way.

### EXAMPLES

### Example 1: Generation and Characterization of Human Antibodies 109 and 186

Eight Trianni human IgG transgenic mice (https://www.nature.com/articles/d43747-020-00174-5.pdf) were immunized using a mammalian expression vector containing the cDNA of canine IL-31 receptor alpha (IL-31RA) (XP_013963900.1) using a proprietary method of DNA immunization. Serum titer response was assessed by incubating a dilution series with cells transiently expressing canine IL-31 receptor alpha (IL-31RA). Binding of antigen-specific antibodies to the cells was then detected using a fluorescently labeled secondary antibody via high-throughput, plate-based flow cytometry. Following 14 weeks of immunization, all eight mice had substantial titers against canine IL-3IRA.

Lymph nodes, spleen and bone marrow were collected from immunized mice with significant serum titers against canine IL-31 receptor alpha (IL-31RA). The cells from all tissues were isolated and enriched for plasma cells and the enriched plasma cell suspension was injected into an AbCellera microfluidic screening device.

Single B cells secreting canine IL-31RA-specific antibodies were identified and isolated using a cell-based assay consisting of cells transiently transfected with canine IL-31RA. Binding was detected using a fluorescently labeled secondary antibody specific to mouse Fc. Positive hits were identified using machine vision and recovered using automated robotics-based protocols. Single cell polymerase chain reaction (PCR) and custom molecular biology protocols generated NGS sequencing libraries (MiSeq, Illumina) using automated workstations (Bravo, Agilent). Sequencing data were analyzed using a custom bioinformatics pipeline to yield paired heavy and light chain sequences for each recovered antibody-secreting cell. A total of ˜350,000 cells were screened. 643 binders were recovered from the screening device for sequencing, and 422 high confidence sequences were obtained. 241 unique antibodies were identified and then annotated with the closest germline (V[D]J) genes and degree of somatic hypermutation. Antibodies were considered members of the same clonal family if they shared the same heavy and light V and J genes and had the same CDR3 length. Seventy-two clonal families were identified, and ninety-two antibodies were selected for expression and purification based on diversity and lack of cysteine liabilities. The variable (V[D]J) region of each antibody chain was synthesized and inserted into a mammalian expression vector with a human IgG1 constant domain for the heavy chains or a human kappa or lambda constant domain for the light chains. Heavy and light chain expression vectors were transiently transfected into HEK 293F cells and the antibodies purified with protein A chromatography. The antibodies were formulated in phosphate buffered saline (PBS), pH 7.2. Purified antibodies were quantified by UV absorption spectrophotometry at 280 nm. Ninety-one of the ninety-two antibodies expressed at sufficient levels for characterization of binding and functional activity.

### Example 2: Inhibition of IL-31 Signaling by Anti-Canine IL-31RA Antibodies

Anti-canine IL-3IRA antibodies were tested for their ability to block IL-31 signaling through IL-3IRA using canine DH-82 cells. The level of pSTAT3 in the DH82 cells was monitored to detect IL-3IRA signaling. The AlphaLISA SureFire Ultra p-STAT3 assay kit (Perkin-Elmer, Cat #ALSU-PST3-A-HV) was used to quantitatively detect phospho-STAT3 levels in the lysates of treated DH82 cells.

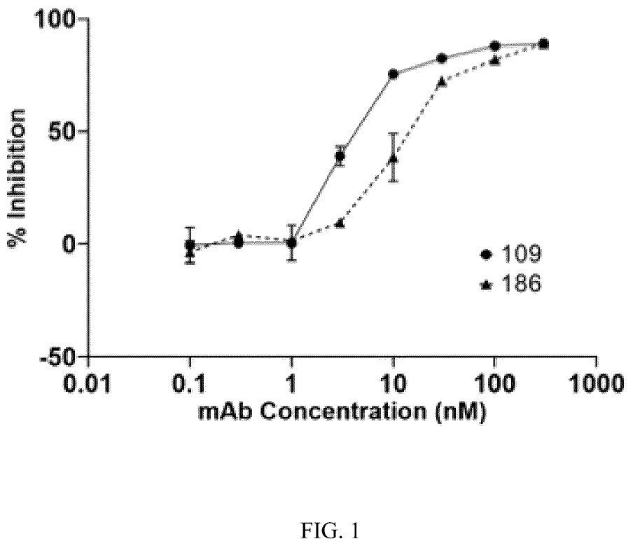

Canine DH-82 canine cells (ATCC CRL-10389, Lot:70031117) were cultured in Eagles' Minimum Essential Medium (EMEM) with 15% fetal bovine serum. On day 1 of the assay, cells were seeded into a 96-well flat-bottom tissue culture plate at 40,000 cells/well with 100 μl medium and placed at 37° C. in 5% CO₂ overnight until stimulation. On day 2, the cells were stimulated with 50 μL of a 30 ng/mL canine interferon-7 solution (IFN-7, R&D Systems, 781-CG-050). On day 3 of the assay, 130 μL of medium was slowly removed from each well and then 200 μL of serum-free EMEM was added dropwise and removed. Finally, 180 μL of serum-free EMEM was added in slowly. The cells were then incubated for two hours at 37° C. The medium was carefully removed from the DH-82 cells and replaced with 45 μL of anti-canine IL-31RA antibodies or human IgG1 isotype control (Southern Biotech; Cat #0151K-01) diluted in EMEM for 30 minutes at 37° C. 5 μL of recombinant canine IL-31 (Novus, Cat #NBP2-59591) at 20 μg/mL was added. After 5 minutes at room temperature, all medium was removed from the wells, and the cells were lysed in 50 μL of 1× lysis buffer (provided in the Perkin-Elmer ALSU-PST3-A-HV kit) and agitated as per the manufacturer's protocol. Next, 30 μL of the lysates were then transferred to a 96-well plate (Perkin-Elmer, cat #6002350) for the AlphaLISA SureFire Ultra Assay and the manufacturer's protocol was followed thereafter. The ninety-one antibodies were tested in the functional assay with canine DH-82 cells. The antibodies were tested at 100 nM in duplicate. The top two antibodies for inhibition in this assay were clones 109 and 186 (SEQ ID NOs:1-4). These two antibodies were retested in the functional assay with a dilution series and both antibodies were able to effectively block the IL-31 signaling (FIG. 1).

### Example 3: Binding of Clones 109 and 186 to Canine IL-31RA

The clones 109 and 186 were evaluated for their binding to the extracellular domain of canine IL-31RA fused to murine IgG2A Fc (SEQ ID NO:5) by SPR. Canine IL-31RA-murine IgG2A Fc was generated by transiently transfecting pcDNA3.4 containing Sequence 3 into GenScript HD (High Density) CHO-S cells and purified with MabSelect SuRe LX chromatography. Further purification of the recombinant protein was completed using gel filtration with TSKgel G3000SWxi chromatography. Antibodies 109 and 186 were captured on to the Cytiva sensor chip with a Human Antibody Capture Kit (BR100839). Antibodies were flowed over the anti-human IgG sensor chip at a concentration of 1 μg/ml in 1×HBS-EP+ (Cytiva, BR100826) for 60 seconds at a flow rate of 10 μL/min. Canine IL-31RA-mFc (100 nM, 50 nM, 25 nM, 12.5 nM, 6.25 nM, 3.625 cam nM) solutions in 1×HBS-EP+ was flown over the sensor chips for a contact time of 120 s and a dissociation time of 600 s at a flow rate of 30 μL/min. Double reference-subtracted sensorgrams were fitted to a 1:1 binding model using Biacore T200 Evaluation software and the kinetics of binding are shown below in Table 4.

TABLE 4
Affinity of antibody 109 and 186 for canine IL-31RA

| | ka | kd | KD | Rmax |
|---|---|---|---|---|
| Antibody | (1/Ms) | (1/s) | (M) | (RU) |
| 109 | 2.14E+05 | 1.08E−04 | 5.06E−10 | 153 |
| 186 | 6.52E+04 | 1.18E−04 | 1.81E−09 | 114 |

### Example 4: Caninization of Antibody Clones 109 and 186

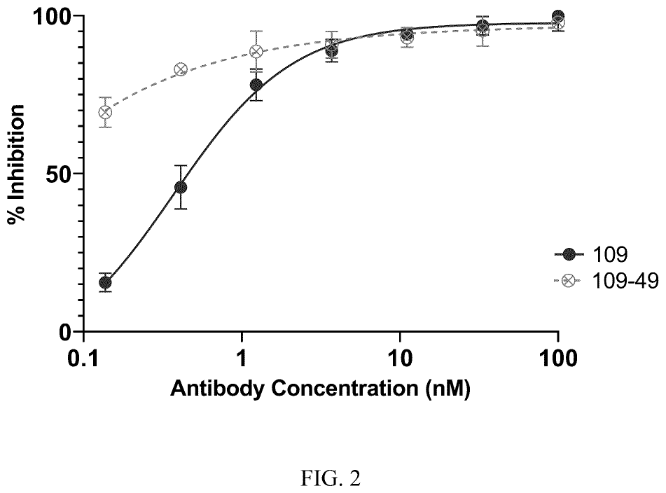

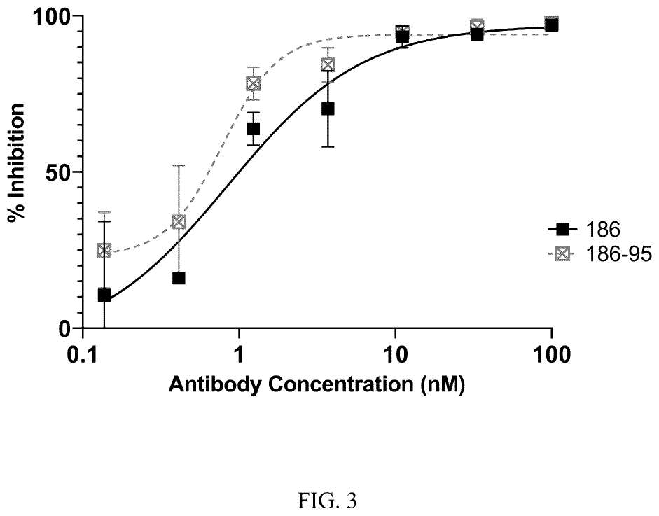

The antibody clones 109 and 186 were caninized by replacing the human frameworks with canine germline frameworks. Using a proprietary informatics method, six heavy chain germline canine frameworks were selected along with fifteen light chain germline frameworks for caninizing antibody clone 109. The same informatics method was used to select three heavy chain germline canine frameworks and three light chain germline canine frameworks for caninizing antibody clone 186. The heavy and light CDRs as defined by the Kabat definition were grafted into the selected heavy and light chain frameworks. The DNA for these caninized constructs were synthesized with the canine IgGB constant and kappa constant domains. In addition, the canine constant domains were fused with the heavy and light chain variable domains of clones 109 and 186 to generate canine chimeric constructs (SEQ ID NOS:6-9). The constructs were cloned into a proprietary mammalian expression vector. Each of the different heavy and light chains were paired and co-transfected into HEK 293 cells and the IgGs in the conditioned medium were purified with MabSelect SuRe protein A resin. The antibodies were buffered exchanged into 20 mM acetate, 136 mM NaCl, pH 5.5. An SPR assay was completed on the caninized antibodies using a Carterra instrument. The antigen which is Canine IL-31RA-murine IgG2A Fc is a dimer and was also aggregated so the apparent affinities were very high because of avidity effects from antigen. Two of the caninized clones with the highest affinities from the SPR experiments were 49 for clone 109 (109-49) and 95 for clone 186 (186-95). The sequences for clones 109-49 and 186-95 are shown in sequences 10-13 of Table 1 (SEQ ID NOs:10-13). Antibody clone 109-49 has the canine framework IGHV3-41 for the heavy chain and IGKV2-20 for the kappa light chain. Antibody clone 186-95 has the canine framework IGHV3-9 for the heavy chain and IGKV4-15 for the light chain. These two caninized clones and the parental canine chimeric antibodies were evaluated for their ability to block IL-31 signaling in canine DH-82 cells using the pSTAT3 protocol described above. The graphs for the antibody inhibition of IL-3IRA are shown in FIGS. 2 and 3.

### Example 5: Removal of Potential Sequence Liabilities from Antibody Clones 109-49 and 186-95

There are potential aspartate isomerization sites and oxidation sites at methionine or tryptophan residues in both antibody clone 109-49 and 186-95. Aspartate isomerization or oxidation in the CDRs can potentially reduce the potency and the stability of an antibody (Xu et al., 2019. MABS, 11:239-264). Below in Table 5 are the IMGT CDR residues for both antibodies, and underlined, bold residues are potential sequence liabilities.

TABLE 5
CDR sequences for caninized antibodies 109-49 and 186-95

| Caninized | | | | | | |
|---|---|---|---|---|---|---|
| Antibody | HCDR1 | HCDR2 | HCDR3 | LCDR1 | LCDR2 | LCDR3 |
| 109-49 | GFTFDDYG | INWAGGT | ARESPLRLGDLGG | QSVSSRY | GTS | QQYDNSPRT |
| | (SEQ ID | (SEQ ID | DYFYYYYYMD | (SEQ ID | | (SEQ ID NO: 33) |
| | NO: 29) | NO: 30) | (SEQ ID NO: 31) | NO: 32) | | |
| 186-95 | GFTFSSYA | ISGSGNST | ATQVVYYFKMDV | QSVSSN | GAS | QQYYNWPPFT |
| | (SEQ ID | (SEQ ID | (SEQ ID NO: 36) | (SEQ ID | | (SEQ NO: 38) |
| | NO: 34) | NO: 35) | | NO: 37) | | |

For antibody clone 109-49, the heavy chain variants W53Y, W53F, M118L, and M118I and the light chain variants N94Q and S95L were generated to identify residues that don't reduce the potency of the antibody and remove the potential liability (see Table 2) (SEQ ID NOs:14-17 and 19-20). The individual variants along with the wild-type 109-49 heavy and light chains were synthesized and subcloned into the mammalian expression vector pcDNA 3.4 (ThermoFisher). The different heavy and light chain combinations were co-transfected into ExpiCHO cells using the ExpiCHO transfection kit (ThermoFisher; Cat. #A29133). Seven days following the transient transfection, the IgG in the conditioned medium was purified using MabSelect SuRe chromatography. The antibodies were buffer-exchanged into PBS, pH 7.4. The relative affinity of the variants was determined by SPR on a Biacore T200. The antibody was captured using a CM5 Series S chip amine coupled with an anti-dog H+L antibody (Jackson Immunoresearch). Canine IL-31RA-mFc (100 nM, 50 nM, 25 nM, 12.5 nM, 6.25 nM, 3.625 nM) solutions in 1×HBS-EP+ was flown over the sensor chip for a contact time of 120 s and a dissociation time of 600 s at a flow rate of 30 L/min. Double reference-subtracted sensorgrams were fitted to a 1:1 binding model using Biacore T200 Evaluation software. The canine IL-31RA antigen was both a dimer and aggregated so there were avidity effects with the measured affinities. For these experiments, the relative affinity of the variants is reported using the following scale: 3 pM→100 pM=3 stars; 101 pM→1 nM=2 stars; and 1.1 nM→20 nM=1 star and is shown in Table 6. Following the initial results with the single variants, a heavy chain containing the W53F variant and the M118I variant was synthesized and subcloned into pcDNA 3.4 (SEQ ID NO:18). The same methods as described above for expression and purification of the variant along with measuring the relative affinity were used to determine the relative affinity of the W53F/M118I heavy chain variant combined with the S95A light chain and the N94Q light chain variants.

TABLE 6
Relative affinities of antibody 109-49 variants.

| HC | LC | | | | Relative |
|---|---|---|---|---|---|
| Variants | Variants | HCDR2 | HCDR3 | LCDR3 | Affinity |
| Wild-type | Wild-type | INWAGGT | ARESPLRLGDLGGDYFYYYYYMD | QQYDNSPRT | *** |
| | | (SEQ ID NO: 30) | (SEQ ID NO: 31) | 410 (SEQ ID | |
| | | | | NO: 33) | |
| W53Y | Wild-type | INYAGGT | ARESPLRLGDLGGDYFYYYYYMD | QQYDNSPRT | * |
| | | (SEQ ID NO: 39) | (SEQ ID NO: 31) | (SEQ ID NO: 33) | |
| W53F | Wild-type | INFAGGT | ARESPLRLGDLGGDYFYYYYYMD | QQYDNSPRT | * |
| | | (SEQ ID NO: 40) | (SEQ ID NO: 31) | (SEQ ID NO: 33) | |
| M118L | Wild-type | INWAGGT | ARESPLRLGDLGGDYFYYYYYLD | QQYDNSPRT | * |
| | | (SEQ ID NO: 30) | (SEQ ID NO: 41) | (SEQ ID NO: 33) | |
| M118I | Wild-type | INWAGGT | ARESPLRLGDLGGDYFYYYYYID | QQYDNSPRT | *** |
| | | (SEQ ID NO: 30) | (SEQ ID NO: 42) | (SEQ ID NO: 33) | |
| Wild-type | N94Q | INWAGGT | ARESPLRLGDLGGDYFYYYYYMD | QQYDQSPRT | ** |
| | | (SEQ ID NO: 30) | (SEQ ID NO: 31) | (SEQ ID NO: 43) | |
| Wild-type | S95A | INWAGGT | ARESPLRLGDLGGDYFYYYYYMD | QQYDNAPRT | *** |
| | | (SEQ ID NO: 30) | (SEQ ID NO: 31) | (SEQ ID NO: 44) | |
| M118I | S95A | INWAGGT | ARESPLRLGDLGGDYFYYYYYID | QQYDNAPRT | *** |
| | | (SEQ ID NO: 30) | (SEQ ID NO: 42) | (SEQ ID NO: 44) | |
| M118I | N94Q | INWAGGT | ARESPLRLGDLGGDYFYYYYYID | QQYDQSPRT | *** |
| | | (SEQ ID NO: 30) | (SEQ ID NO: 42) | (SEQ ID NO: 43) | |
| W53F- | S95A | INFAGGT | ARESPLRLGDLGGDYFYYYYYID | QQYDNAPRT | * |
| M118I | | (SEQ ID NO: 40) | (SEQ ID NO: 42) | (SEQ ID NO: 44) | |
| W53F- | N94Q | INFAGGT | ARESPLRLGDLGGDYFYYYYYID | QQYDQSPRT | * |
| M118I | | (SEQ ID NO: 40) | (SEQ ID NO: 42) | (SEQ ID NO: 43) | |

For antibody clone 186-95, the heavy chain variants N56Q, S57A, M106L, and M106I and the light chain variants W94Y and W94F were generated to identify residues that don't reduce the potency of the antibody and remove the potential liability (see Table 3) (SEQ ID NOs:21-24, 26 and 27). Using the same methods as described for the 109-49 variants, the 186-95 variants were synthesized, expressed, purified and the affinities to canine IL-3 IRA were determined with SPR. Similar to the Biacore experiments with the 109-49 variants, the canine-IL-31RA-murine Fc protein resulting in avidity effects and artificially high apparent affinities. The relative affinities for the affinities are reported using the same scale as described for the 109-49 variants and shown in Table 7. Following the initial results with the single variants, a 186-95 heavy chain containing the N56Q variant and the M106L variant was synthesized and subcloned into pcDNA 3.4 (SEQ ID NO:25). The same methods as described above for expression and purification of the variant along with measuring the relative affinity were used to determine the relative affinity of the N56Q/M106L heavy chain variant combined with the W94F light chain and the W94Y light chain variants.

TABLE 7
Relative affinities of antibody 186-95 variants

| HC | LC | | | | Relative |
|---|---|---|---|---|---|
| Variants | Variants | HCDR2 | HCDR3 | LCDR3 | Affinity |
| Wild-type | Wild-type | ISGSGNST | ATQVVYYFKMDV | QQYYNWPPFT | *** |
| | | (SEQ IE NO: 35) | (SEQ IE NO: 36) | (SEQ IE NO: 38) | |
| N56Q | Wild-type | ISGSGQST | ATQVVYYFKMDV | QQYYNWPPFT | *** |
| | | (SEQ IE NO: 45) | (SEQ IE NO: 36) | (SEQ IE NO: 38) | |
| S57A | Wild-type | ISGSGNAT | ATQVVYYFKMDV | QQYYNWPPFT | *** |
| | | (SEQ IE NO: 46) | (SEQ IE NO: 36) | (SEQ IE NO: 38) | |
| M106L | Wild-type | ISGSGNST | ATQVVYYFKLDV | QQYYNWPPFT | *** |
| | | (SEQ IE NO: 35) | (SEQ IE NO: 47) | (SEQ IE NO: 38) | |
| M106I | Wild-type | ISGSGNST | ATQVVYYFKIDV | QQYYNWPPFT | *** |
| | | (SEQ IE NO: 35) | (SEQ IE NO: 48) | (SEQ IE NO: 38) | |
| Wild-type | W94Y | ISGSGNST | ATQVVYYFKMDV | QQYYNYPPFT | *** |
| | | (SEQ IE NO: 35) | (SEQ IE NO: 36) | (SEQ IE NO: 49) | |
| Wild-type | W94F | ISGSGNST | ATQVVYYFKMDV | QQYYNFPPFT | *** |
| | | (SEQ IE NO: 35) | (SEQ IE NO: 36) | (SEQ IE NO: 50) | |
| N56Q-M106L | W94F | ISGSGQST | ATQVVYYFKLDV | QQYYNFPPFT | *** |
| | | (SEQ IE NO: 45) | (SEQ IE NO: 47) | (SEQ IE NO: 50) | |
| N56Q-M106L | W94Y | ISGSGQST | ATQVVYYFKLDV | QQYYNYPPFT | *** |
| | | (SEQ IE NO: 45) | (SEQ IE NO: 47) | (SEQ IE NO: 49) | |

To reduce the avidity effects from using the canine IL-31RA-mouse Fc construct in SPR experiments, a monomeric construct was generated which contains amino acids 20-308 of the canine IL-31IRA extracellular domain (SEQ ID NO:28). This construct has the AviTag sequence (GLNDIFEAQKIEWHE (SEQ ID NO:51)) followed by an 8×His(SEQ ID NO:52) tag to facilitate purification. GenScript HD (High Density) CHO-S cells were transiently transfected with pcDNA 3.4 containing Sequence 28 and purified with HisTrap FF chromatography. Further purification of the recombinant protein was completed using gel filtration with Cytiva HiLoad 26/600 Superdex chromatography to isolate the monomeric IL-31RA species. Two of the best caninized clones with their potential sequence liabilities removed are 109-49 containing the M118I and S95A variants which is referred to as 109-49_IA (SEQ ID NOs:17 and 20) and 186-95 containing the N56Q, M106L, and W94F variants which is referred to as 186-95_QLF (SEQ ID NOs:25 and 27). These clones were evaluated in an SPR experiment using the same conditions as described for the caninized clones except the analyte is canine IL-31RA (20-308). The affinity of 109-49_IA and 186-95_QLF for canine IL-31RA (20-308) are shown below in Table 8.

TABLE 8
Affinities of 109-49_IA and
186-95_QLF for canine IL-31RA (20-308)

| Caninized | ka | kd | KD | Rmax |
|---|---|---|---|---|
| Antibody | (1/Ms) | (1/s) | (M) | (RU) |
| 109-49_IA | 5.44E+4 | 7.09E−05 | 1.31E−09 | 83 |
| 186-95_QLF | 6.98E+4 | 1.61E−04 | 2.31E−09 | 112 |

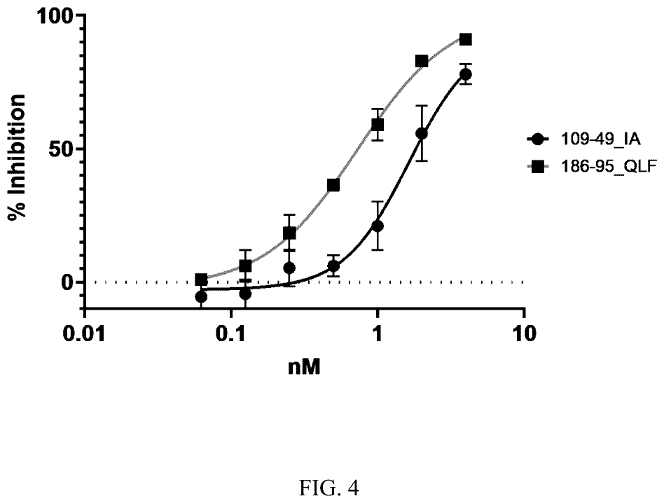

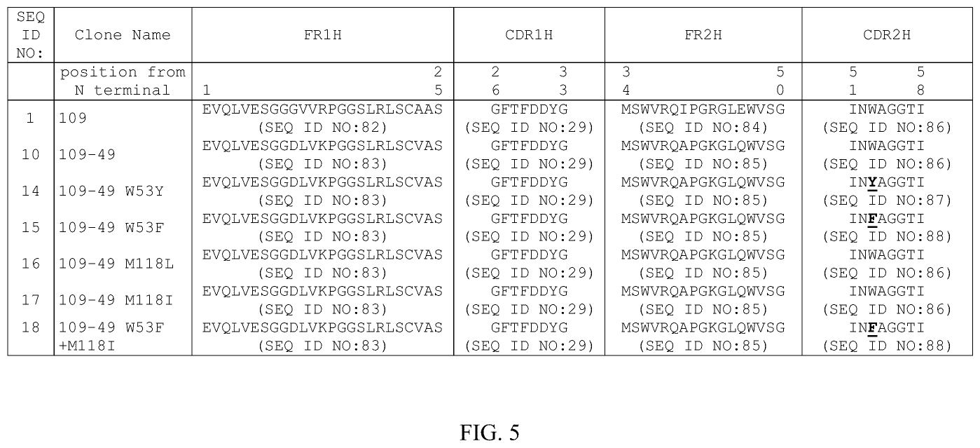

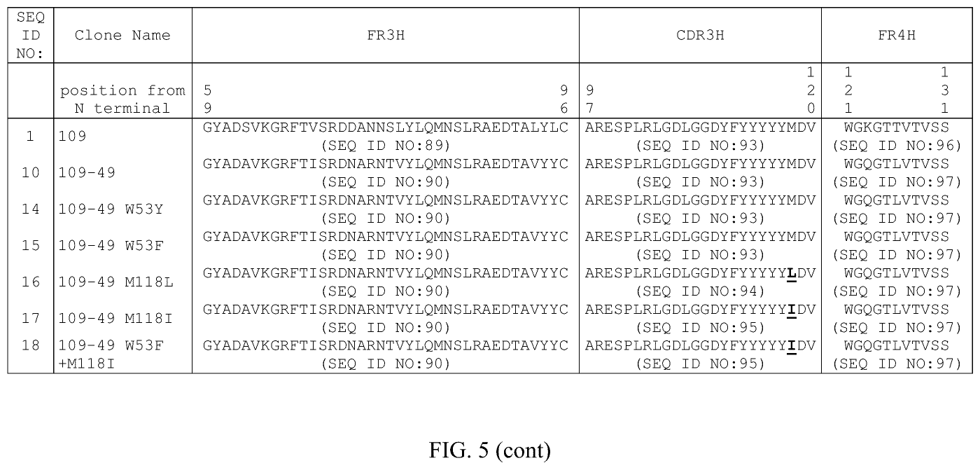

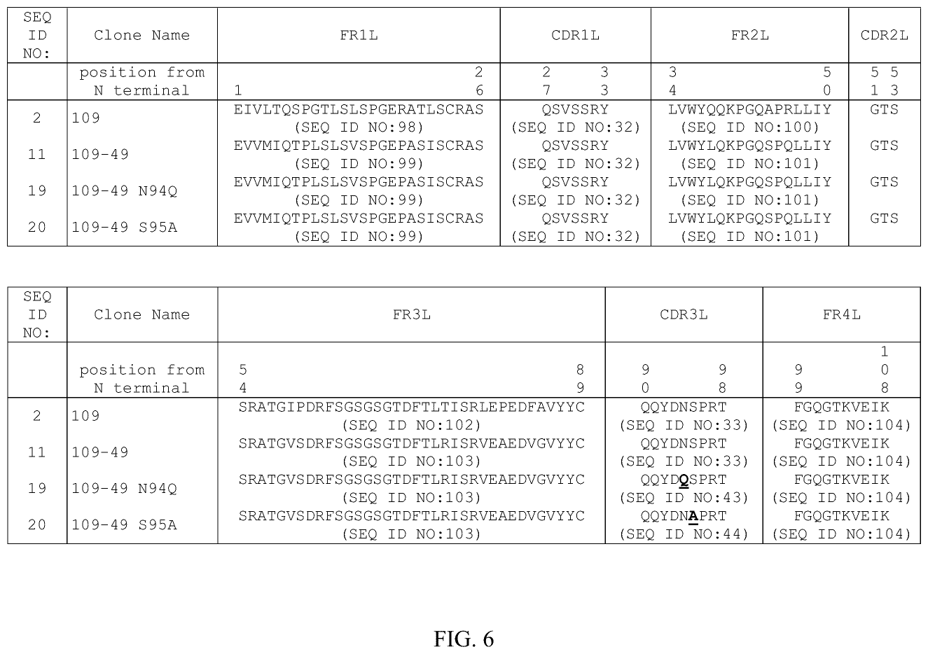

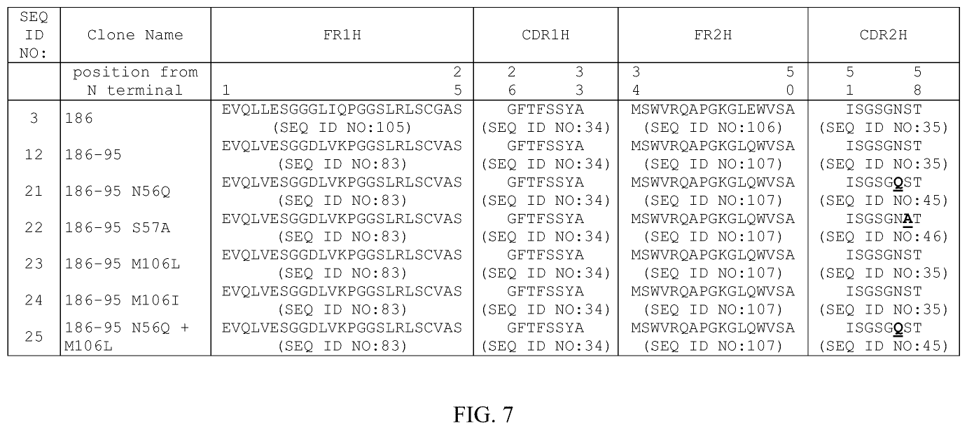

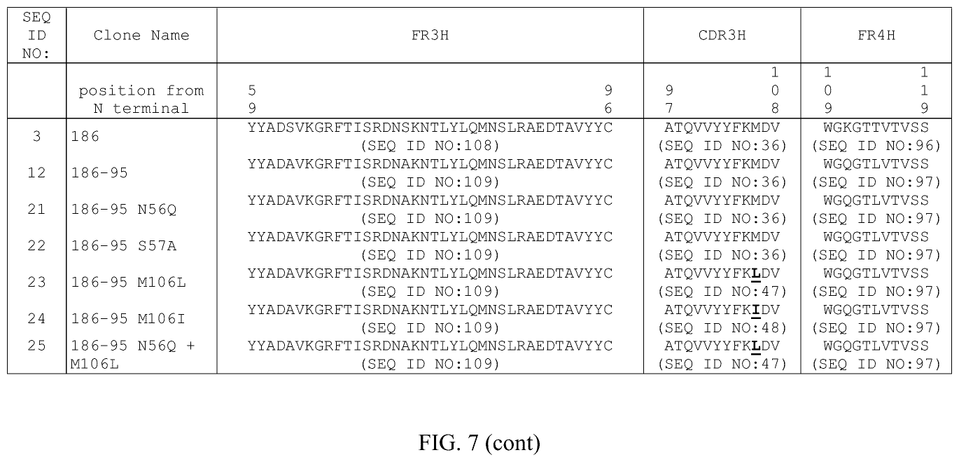

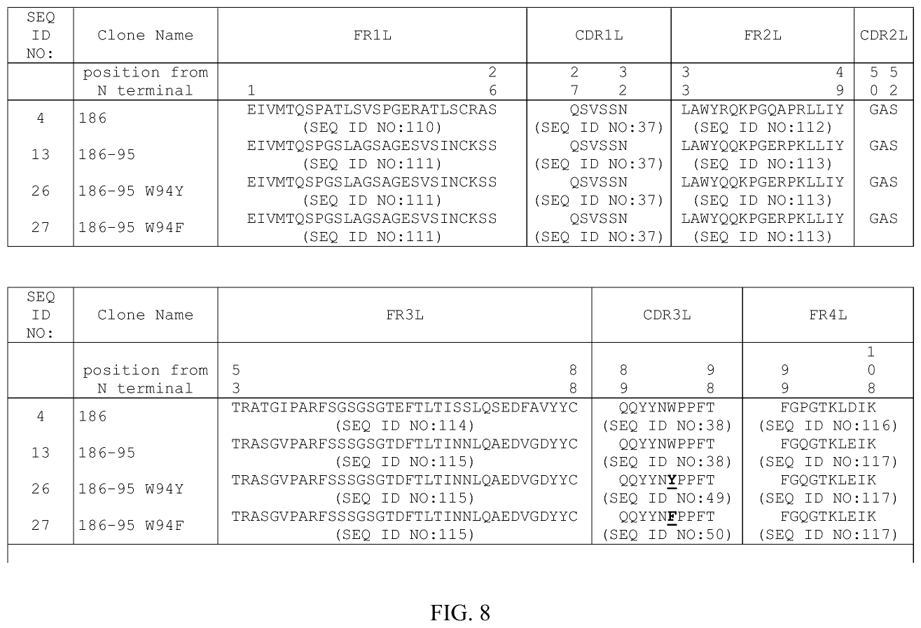

Antibody clones 109-49_IA and 186-95_QLF were evaluated for their ability to block IL-31 signaling in canine DH-82 cells using the pSTAT3 protocol described above for the caninized antibody clones 109 and 186 (FIG. 4).

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. An antigen binding protein that specifically binds to interleukin-31 receptor alpha (IL-31RA), which comprises:
(a) a heavy chain complementarity determining region 1 (HCDR1) comprising X₂₆X₂₇X₂₈X₂₉X₃₀X₃₁X₃₂X₃₃ (SEQ ID NO:53), wherein X₂₆ comprises A, G, I, L, M, W, F, P, or V, X₂₇ comprises A, G, I, L, M, W, F, P, V, or Y, X₂₈ comprises C, S, T, Y, N, or Q, X₂₉ comprises A, G, I, L, M, W, F, P, V, or Y, X₃₀ comprises D or E, X₃₁ comprises D or E, X₃₂ comprises C, S, T, Y, N, or Q, and X₃₃ comprises A, G, I, L, M, W, F, P, or V;
(b) a heavy chain complementarity determining region 2 (HCDR2) comprising X₅₁X₅₂X₅₃X₅₄X₅₅X₅₆X₅₇ (SEQ ID NO:54), wherein X₅₁ comprises A, G, I, L, M, W, F, P, or V, X₅₂ comprises C, S, T, Y, N, or Q, X₅₃ comprises A, I, L, M, F, P, W, Y, or V, X₅₄ comprises A, G, I, L, M, W, F, P, or V, X₅₅ comprises A, G, I, L, M, W, F, P, or V, X₅₆ comprises A, G, I, L, M, W, F, P, or V, and X₅₇ comprises C, S, T, Y, N, or Q;
(c) a heavy chain complementarity determining region 3 (HCDR3) comprising X₉₇X₉₈X₉₉X₁₀₀X₁₀₁X₁₀₂X₁₀₃X₁₀₄X₁₀₅X₁₀₆X₁₀₇X₁₀₈X₁₀₉X₁₁₀X₁₁₁X₁₁₂X₁₁₃X₁₁₄X₁₁₅X₁₁₆X₁₁₇X₁₁₈X₁₁₉ (SEQ ID NO:55), wherein X₉₇ comprises A, G, I, L, M, W, F, P, or V, X₉₈ comprises H, K, or R, X₉₉ comprises E or D, X₁₀₀ comprises C, S, T, Y, N, or Q, X₁₀₁ comprises A, G, I, L, M, W, F, P, or V, X₁₀₂ comprises A, G, I, L, M, W, F, P, or V, X₁₀₃ comprises H, K, or R, X₁₀₄ comprises A, G, I, L, M, W, F, P, or V, X₁₀₅ comprises A, G, I, L, M, W, F, P, or V, X₁₀₆ comprises E or D, X₁₀₇ comprises A, G, I, L, M, W, F, P, or V, X₁₀₈ comprises A, G, I, L, M, W, F, P, or V, X₁₀₉ comprises A, G, I, L, M, W, F, P, or V, X₁₁₀ comprises E or D, X₁₁₁ comprises C, S, T, Y, N, or Q, X₁₁₂ comprises A, G, I, L, M, W, F, P, V, or Y, X₁₁₃ comprises an C, S, T, Y, N, or Q, X₁₁₄ comprises C, S, T, Y, N, or Q, X₁₁₅ comprises C, S, T, Y, N, or Q, X₁₁₆ comprises C, S, T, Y, N, or Q, X₁₁₇ comprises C, S, T, Y, N, or Q, X₁₁₈ comprises A, G, I, L, M, W, F, P, or V, and X₁₁₉ comprises E or D;
(d) a light chain complementarity determining region 1 (LCDR1) comprising X₂₇X₂₈X₂₉X₃₀X₃₁X₃₂X₃₃ (SEQ ID NO:56), wherein X₂₇ comprises C, S, T, Y, N, or Q, X₂₈ comprises C, S, T, Y, N, or Q, X₂₉ comprises A, G, I, L, M, W, F, P, or V, X₃₀ comprises C, S, T, Y, N, or Q, X₃₁ comprises C, S, T, Y, N, or Q, X₃₂ comprises H, K, or R, and X₃₃ comprises C, S, T, Y, N, or Q;
(e) a light chain complementarity determining region 2 (LCDR2) comprising X₅₁X₅₂X₅₃, wherein X₅₁ comprises A, G, I, L, M, W, F, P, or V, X₅₂ comprises C, S, T, Y, N, or Q, and X₅₃ comprises C, S, T, Y, N, or Q; and
(f) a light chain complementarity determining region 3 (LCDR3) comprising X₉₀X₉₁X₉₂X₉₃X₉₄X₉₅X₉₆X₉₇X₉₈ (SEQ ID NO:57), wherein X₉₀ comprises C, S, T, Y, N, or Q, X₉₁ comprises C, S, T, Y, N, or Q, X₉₂ comprises C, S, T, Y, N, or Q, X₉₃ comprises E or D, X₉₄ comprises C, S, T, Y, N, or Q, X₉₅ comprises C, L, S, T, Y, N, or Q, X₉₆ comprises A, G, I, L, M, W, F, P, or V, X₉₇ comprises H, K, or, R, and X₉₈ comprises C, S, T, Y, N, or Q.

2. The antigen-binding protein of claim 1, wherein:
(a) HCDR1 comprises GFTFDDYG (SEQ ID NO:29) or differs at no more than one or two positions; and/or
(b) HCDR2 comprises INX₅₃AGGT (SEQ ID NO:58), wherein X₅₃ comprises F, W, or Y; and/or
(c) HCDR3 comprises ARESPLRLGDLGGDYFYYYYYX₁₁₈D (SEQ ID NO:59), wherein X₁₁₈ comprises I, L, or M; and/or
(d) LCDR1 comprises QSVSSRY (SEQ ID NO:32) or differs at no more than one or two positions; and/or
(e) LCDR2 comprises GTS or differs at no more than one or two positions; and/or
(f) LCDR3 comprises QQYDX₉₄X₉₅PRT (SEQ ID NO:60), wherein X₉₄ comprises N, or Q, and X₉₅ comprises S or L.

3. The antigen-binding protein of claim 1, which comprises the heavy chain variable domain CDRs of SEQ ID NO:17 and the light chain variable domain CDRs of SEQ ID NO:20.

4. The antigen binding protein of claim 3, wherein the antigen binding protein comprises a heavy chain variable domain at least 80%, at least 85%, at least 90%, at least 95%, at least 97%, at least 98%, at least 99%, or 100% identical to SEQ ID NO:17, and a light chain variable domain at least 80%, at least 85%, at least 90%, at least 95%, at least 97%, at least 98%, at least 99%, or 100% identical to SEQ ID NO:20.

5. The antigen binding protein of claim 1, wherein the heavy chain variable domain comprises W53F, W53Y, Ml 181, or M118L and/or the light chain variable domain comprises N94Q or S95L.

6. The antigen binding protein of claim 1, wherein the heavy chain variable domain comprises the IMGT CDRs of SEQ ID NO:1, SEQ ID NO:10, SEQ ID NO:14, SEQ ID NO:15, SEQ ID NO:16, or SEQ ID NO:17, and wherein the light chain variable domain comprises the IMGT CDRs of SEQ ID NO:2, SEQ ID NO: 11, SEQ ID NO:19, or SEQ ID NO:20.

7. The antigen binding protein of claim 1, wherein the antigen binding protein comprises a heavy chain variable domain at least 80%, at least 85%, at least 90%, at least 95%, at least 97%, at least 98%, at least 99%, or 100% identical to a heavy chain variable domain of SEQ ID NO:1, SEQ ID NO:10, SEQ ID NO:14, SEQ ID NO:15, SEQ ID NO:16, or SEQ ID NO:17, and a light chain variable domain at least 80%, at least 85%, at least 90%, at least 95%, at least 97%, at least 98%, at least 99%, or 100% identical to a light chain variable domain of SEQ ID NO:2, SEQ ID NO:11, SEQ ID NO:19, or SEQ ID NO:20.

8. An antigen binding protein that specifically binds to IL-3IRA, which comprises:
(a) a heavy chain complementarity determining region 1 (HCDR1) comprising X₂₆X₂₇X₂₈X₂₉X₃₀X₃₁X₃₂X₃₃ (SEQ ID NO:61), wherein X₂₆ comprises A, G, I, L, M, W, F, P, or V, X₂₇ comprises A, G, I, L, M, W, F, P, V, or Y, X₂₈ comprises C, S, T, Y, N, or Q, X₂₉ comprises A, G, I, L, M, W, F, P, V, of Y, X₃₀ comprises C, S, T, Y, N, or Q, X₃₁ comprises C, S, T, Y, N, or Q, X₃₂ comprises C, S, T, Y, N, or Q, and X₃₃ comprises A, G, I, L, M, W, F, P, or V;
(b) a heavy chain complementarity determining region 2 (HCDR2) comprising X₅₁X₅₂X₅₃X₅₄X₅₅X₅₆X₅₇ (SEQ ID NO:62), wherein X₅₁ comprises A, G, I, L, M, W, F, P, or V, X₅₂ comprises C, S, T, Y, N, or Q, X₅₃ comprises A, G, I, L, M, W, F, P, or V, X₅₄ comprises C, S, T, Y, N, or Q, X₅₅ comprises A, G, I, L, M, W, F, P, or V, X₅₆ comprises C, S, T, Y, N, or Q, and X₅₇ comprises A, C, S, T, Y, N, or Q;
(c) a heavy chain complementarity determining region 3 (HCDR3) comprising X₉₇X₉₈X₉₉X₁₀₀X₁₀₁X₁₀₂X₁₀₃X₁₀₄X₁₀₅X₁₀₆X₁₀₇ (SEQ ID NO:63), wherein X₉₇ comprises A, G, I, L, M, W, F, P, or V, X₉₈ comprises C, S, T, Y, N, or Q, X₉₉ comprises C, S, T, Y, N, or Q, X₁₀₀ comprises A, G, I, L, M, W, F, P, or V, X₁₀₁ comprises A, G, I, L, M, W, F, P, or V, X₁₀₂ comprises C, S, T, Y, N, or Q, X₁₀₃ comprises C, S, T, Y, N, or Q, X₁₀₄ comprises A, G, I, L, M, W, F, P, V, or Y, X₁₀₅ comprises H, K, or R, X₁₀₆ comprises A, G, I, L, M, W, F, P, or V, and X₁₀₇ comprises E or D;
(d) a light chain complementarity determining region 1 (LCDR1) comprising X₂₇X₂₈X₂₉X₃₀X₃₁X₃₂ (SEQ ID NO:64) wherein X₂₇ comprises C, S, T, Y, N, or Q, X₂₈ comprises C, S, T, Y, N, or Q, X₂₉ comprises A, G, I, L, M, V, W, F, P, or V, X₃₀ comprises C, S, T, Y, N, or Q, X₃₁ comprises C, S, T, Y, N, or Q, and X₃₂ comprises C, S, T, Y, N, or Q;
(e) a light chain complementarity determining region 2 (LCDR2) comprising X₅₀X₅₁X₅₂, wherein X₅₀ comprises A, G, I, L, M, W, F, P, or V, X₅₁ comprises A, G, I, L, M, W, F, P, or V, and X₅₂ comprises C, S, T, Y, N, or Q; and
(f) a light chain complementarity determining region 3 (LCDR3) comprising X₈₉X₉₀X₉₁X₉₂X₉₃X₉₄X₉₅X₉₆X₉₇X₉₈ (SEQ ID NO:65), wherein X₈₉ comprises C, S, T, Y, N, or Q, X₉₀ comprises C, S, T, Y, N, or Q, X₉₁ comprises A, G, I, L, M, W, F, P, V, or Y, X₉₂ comprises A, G, I, L, M, W, F, P, V, or Y, X₉₃ comprises C, S, T, Y, N, or Q, X₉₄ comprises A, I, L, M, F, P, W, Y, or V, X₉₅ comprises A, G, I, L, M, W, F, P, or V, X₉₆ comprises A, G, I, L, M, W, F, P, or V, X₉₇ comprises A, G, I, L, M, W, F, P, V, or Y, and X₉₈ comprises C, S, T, Y, N, or Q.

9. The antigen-binding protein of claim 8, wherein:
(a) HCDR1 comprises GFTFSSYA (SEQ ID NO:34); and/or
(b) HCDR2 comprise ISGSGX₅₆X₅₇T (SEQ ID NO:66), wherein X₅₆ comprises N, or Q; and X₅₇ comprises A or S; and/or
(c) HCDR3 comprises ATQVVYYFKX₁₀₆DV (SEQ ID NO:67), wherein X₁₀₆ comprises I, L, or M; and/or
(d) LCDR1 comprises QSVSSN (SEQ ID NO:37); and/or
(e) LCDR2 comprises GAS; and/or
(f) LCDR3 comprises QQYYNX₉₄PPFT (SEQ ID NO:68), wherein X₉₄ comprises F, Y, or W.

10. The antigen binding protein of claim 8, which comprises the heavy chain variable domain CDRs of SEQ ID NO:25 and the light chain variable domain CDRs of SEQ ID NO:27.

11. The antigen binding protein of claim 10, wherein the antigen binding protein comprises a heavy chain variable domain at least 80%, at least 85%, at least 90%, at least 95%, at least 97%, at least 98%, at least 99%, or 100% identical to SEQ ID NO:25, and a light chain variable domain at least 80%, at least 85%, at least 90%, at least 95%, at least 97%, at least 98%, at least 99%, or 100% identical to SEQ ID NO:27.

12. The antigen binding protein of claim 8, wherein the heavy chain variable domain comprises N56Q, S57A, M106I, or M106L and/or the light chain variable domain comprises W94F or W94Y.

13. The antigen binding protein of claim 8, wherein the heavy chain variable domain comprises the IMGT CDRs of SEQ ID NO:3, SEQ ID NO:12, SEQ ID NO:21, SEQ ID NO:22, SEQ ID NO:23, SEQ ID NO:24, or SEQ ID NO:25, and wherein the light chain variable domain comprises the IMGT CDRs of SEQ ID NO:4, SEQ ID NO:13, SEQ ID NO:26, or SEQ ID NO:27.

14. The antigen binding protein of claim 8, wherein the antigen binding protein comprises a heavy chain variable domain at least 80%, at least 85%, at least 90%, at least 95%, at least 97%, at least 98%, at least 99%, or 100% identical to a heavy chain variable domain of SEQ ID NO:3, SEQ ID NO:12, SEQ ID NO:21, SEQ ID NO:22, SEQ ID NO:23, SEQ ID NO:24, or SEQ IDNO:25, and a light chain variable domain at least 80%, at least 85%, at least 90%, at least 95%, at least 97%, at least 98%, at least 99%, or 100% identical to a light chain variable domain of SEQ ID NO:4, SEQ ID NO:13, SEQ ID NO:26, or SEQ ID NO:27.

15. An isolated nucleic acid sequence encoding an anti-IL-31RA antigen binding protein of claim 1.

16. A vector that comprises the nucleic acid of claim 15.

17. A recombinant cell which comprises the nucleic acid of claim 15.

18. A cell that expresses the antigen binding protein of claim 1.

19. A method of producing the antigen binding protein of claim 1.

20. A pharmaceutical composition comprising a therapeutically effective amount of the anti-IL-31RA protein of claim 1.

21. A method of suppressing atopic dermatitis in a subject, which comprises administering to the subject a therapeutically effective amount of an anti-IL-31RA protein of claim 1.

22. A method of inhibiting binding of IL-31 to anti-IL-31A in a subject, which comprises administering to the subject a therapeutically effective amount of the anti-IL-31RA antigen binding protein of claim 1.

23. The method of claim 21, wherein the subject comprises a human, a canine, a feline, or an equine.

24. A method of detecting anti-IL-31RA in a sample comprising incubating the sample with an anti-IL-31RA protein of claim 1 and detecting the anti-IL-31RA protein bound to IL-31RA in the sample.


SEQUENCE LISTING (117 sequences)

SEQ ID NO: 1 - protein, 460 aa, synthetic construct
EVQLVESGGGVVRPGGSLRLSCAASGFTFDDYGMSWVRQIPGRGLEWVSGINWAGGTIGYADSVKGRFTVSRDDANNSLYLQMNSLRAEDTALYLCARESPLRLGDLGGDYFYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 215 aa, synthetic construct
EIVLTQSPGTLSLSPGERATLSCRASQSVSSRYLVWYQQKPGQAPRLLIYGTSSRATGIPDRFSGSGSGTDFTLTISRLEPEDFAVYYCQQYDNSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 448 aa, synthetic construct
EVQLLESGGGLIQPGGSLRLSCGASGFTFSSYAMSWVRQAPGKGLEWVSAISGSGNSTYYADSVKGRFTISRDNSKNTLYLQMNSLRAEDTAVYYCATQVVYYFKMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 215 aa, synthetic construct
EIVMTQSPATLSVSPGERATLSCRASQSVSSNLAWYRQKPGQAPRLLIYGASTRATGIPARFSGSGSGTEFTLTISSLQSEDFAVYYCQQYYNWPPFTFGPGTKLDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 758 aa, synthetic construct
MGWSCIILFLVATATGVHSVLPAKPENISCIFYYEENFTCTWSPEKEASYTWYKVKRTYSYGYKSDICSTDNSTRGNHASCSFLPPTITNPDNYTIQVEAQNADGIMKSDITYWNLDAIMKIEPPEIFSVKSVLGIKRMLQIKWIRPVLAPHSSTLKYTLRFRTINSAYWMEVNFTKEDIDRDETYNLTELQAFTEYVMTLRCAPAESMFWSGWSQEKVGTTEEEAPYGLDLWRVLKPAMVDGRRPVQLMWKKATGAPVLEKALGYNIWYFPENNTNLTETVNTTNQTHELYLGGKTYWVYVVSYNSLGESPVATLRIPALNEKTFQCIEAMQACLTQDQLVVEWQSSAPEVDTWMVEWFPDVDSEPSSFSWESVSQARNWTIQKDELKPLWCYNISVYPVLRDRVGQPYSTQAYVQEGIPSAGPVTQADSIGVKTVTITWKEIPKSKRNGFIKNYTIFYQAEDGKEFSKTVNSNILQYRLESLTRRTSYSLQVMASTNAGGTNGTKINFKTLSISVLGGGSGGGSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 6 - protein, 484 aa, synthetic construct
MGWSCIILFLVATATGVHSEVQLVESGGGVVRPGGSLRLSCAASGFTFDDYGMSWVRQIPGRGLEWVSGINWAGGTIGYADSVKGRFTVSRDDANNSLYLQMNSLRAEDTALYLCARESPLRLGDLGGDYFYYYYYMDVWGKGTTVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSPG

SEQ ID NO: 7 - protein, 238 aa, synthetic construct
METDTLLLWVLLLWVPGSTGEIVLTQSPGTLSLSPGERATLSCRASQSVSSRYLVWYQQKPGQAPRLLIYGTSSRATGIPDRFSGSGSGTDFTLTISRLEPEDFAVYYCQQYDNSPRTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 8 - protein, 472 aa, synthetic construct
MGWSCIILFLVATATGVHSEVQLLESGGGLIQPGGSLRLSCGASGFTFSSYAMSWVRQAPGKGLEWVSAISGSGNSTYYADSVKGRFTISRDNSKNTLYLQMNSLRAEDTAVYYCATQVVYYFKMDVWGKGTTVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSPG

SEQ ID NO: 9 - protein, 238 aa, synthetic construct
METDTLLLWVLLLWVPGSTGEIVMTQSPATLSVSPGERATLSCRASQSVSSNLAWYRQKPGQAPRLLIYGASTRATGIPARFSGSGSGTEFTLTISSLQSEDFAVYYCQQYYNWPPFTFGPGTKLDIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 10 - protein, 484 aa, synthetic construct
MGWSCIILFLVATATGVHSEVQLVESGGDLVKPGGSLRLSCVASGFTFDDYGMSWVRQAPGKGLQWVSGINWAGGTIGYADAVKGRFTISRDNARNTVYLQMNSLRAEDTAVYYCARESPLRLGDLGGDYFYYYYYMDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSPG

SEQ ID NO: 11 - protein, 238 aa, synthetic construct
METDTLLLWVLLLWVPGSTGEVVMIQTPLSLSVSPGEPASISCRASQSVSSRYLVWYLQKPGQSPQLLIYGTSSRATGVSDRFSGSGSGTDFTLRISRVEAEDVGVYYCQQYDNSPRTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 12 - protein, 472 aa, synthetic construct
MGWSCIILFLVATATGVHSEVQLVESGGDLVKPGGSLRLSCVASGFTFSSYAMSWVRQAPGKGLQWVSAISGSGNSTYYADAVKGRFTISRDNAKNTLYLQMNSLRAEDTAVYYCATQVVYYFKMDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSPG

SEQ ID NO: 13 - protein, 238 aa, synthetic construct
METDTLLLWVLLLWVPGSTGEIVMTQSPGSLAGSAGESVSINCKSSQSVSSNLAWYQQKPGERPKLLIYGASTRASGVPARFSSSGSGTDFTLTINNLQAEDVGDYYCQQYYNWPPFTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 14 - protein, 484 aa, synthetic construct
MGWSCIILFLVATATGVHSEVQLVESGGDLVKPGGSLRLSCVASGFTFDDYGMSWVRQAPGKGLQWVSGINYAGGTIGYADAVKGRFTISRDNARNTVYLQMNSLRAEDTAVYYCARESPLRLGDLGGDYFYYYYYMDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSPG

SEQ ID NO: 15 - protein, 484 aa, synthetic construct
MGWSCIILFLVATATGVHSEVQLVESGGDLVKPGGSLRLSCVASGFTFDDYGMSWVRQAPGKGLQWVSGINFAGGTIGYADAVKGRFTISRDNARNTVYLQMNSLRAEDTAVYYCARESPLRLGDLGGDYFYYYYYMDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSPG

SEQ ID NO: 16 - protein, 484 aa, synthetic construct
MGWSCIILFLVATATGVHSEVQLVESGGDLVKPGGSLRLSCVASGFTFDDYGMSWVRQAPGKGLQWVSGINWAGGTIGYADAVKGRFTISRDNARNTVYLQMNSLRAEDTAVYYCARESPLRLGDLGGDYFYYYYYLDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSPG

SEQ ID NO: 17 - protein, 484 aa, synthetic construct
MGWSCIILFLVATATGVHSEVQLVESGGDLVKPGGSLRLSCVASGFTFDDYGMSWVRQAPGKGLQWVSGINWAGGTIGYADAVKGRFTISRDNARNTVYLQMNSLRAEDTAVYYCARESPLRLGDLGGDYFYYYYYIDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSPG

SEQ ID NO: 18 - protein, 484 aa, synthetic construct
MGWSCIILFLVATATGVHSEVQLVESGGDLVKPGGSLRLSCVASGFTFDDYGMSWVRQAPGKGLQWVSGINFAGGTIGYADAVKGRFTISRDNARNTVYLQMNSLRAEDTAVYYCARESPLRLGDLGGDYFYYYYYIDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSPG

SEQ ID NO: 19 - protein, 238 aa, synthetic construct
METDTLLLWVLLLWVPGSTGEVVMIQTPLSLSVSPGEPASISCRASQSVSSRYLVWYLQKPGQSPQLLIYGTSSRATGVSDRFSGSGSGTDFTLRISRVEAEDVGVYYCQQYDQSPRTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 20 - protein, 238 aa, synthetic construct
METDTLLLWVLLLWVPGSTGEVVMIQTPLSLSVSPGEPASISCRASQSVSSRYLVWYLQKPGQSPQLLIYGTSSRATGVSDRFSGSGSGTDFTLRISRVEAEDVGVYYCQQYDNAPRTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 21 - protein, 472 aa, synthetic construct
MGWSCIILFLVATATGVHSEVQLVESGGDLVKPGGSLRLSCVASGFTFSSYAMSWVRQAPGKGLQWVSAISGSGQSTYYADAVKGRFTISRDNAKNTLYLQMNSLRAEDTAVYYCATQVVYYFKMDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSPG

SEQ ID NO: 22 - protein, 472 aa, synthetic construct
MGWSCIILFLVATATGVHSEVQLVESGGDLVKPGGSLRLSCVASGFTFSSYAMSWVRQAPGKGLQWVSAISGSGNATYYADAVKGRFTISRDNAKNTLYLQMNSLRAEDTAVYYCATQVVYYFKMDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSPG

SEQ ID NO: 23 - protein, 472 aa, synthetic construct
MGWSCIILFLVATATGVHSEVQLVESGGDLVKPGGSLRLSCVASGFTFSSYAMSWVRQAPGKGLQWVSAISGSGNSTYYADAVKGRFTISRDNAKNTLYLQMNSLRAEDTAVYYCATQVVYYFKLDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSPG

SEQ ID NO: 24 - protein, 472 aa, synthetic construct
MGWSCIILFLVATATGVHSEVQLVESGGDLVKPGGSLRLSCVASGFTFSSYAMSWVRQAPGKGLQWVSAISGSGNSTYYADAVKGRFTISRDNAKNTLYLQMNSLRAEDTAVYYCATQVVYYFKIDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSPG

SEQ ID NO: 25 - protein, 472 aa, synthetic construct
MGWSCIILFLVATATGVHSEVQLVESGGDLVKPGGSLRLSCVASGFTFSSYAMSWVRQAPGKGLQWVSAISGSGQSTYYADAVKGRFTISRDNAKNTLYLQMNSLRAEDTAVYYCATQVVYYFKLDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQLAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICYVMHEALHNHYTQESLSHSPG

SEQ ID NO: 26 - protein, 238 aa, synthetic construct
METDTLLLWVLLLWVPGSTGEIVMTQSPGSLAGSAGESVSINCKSSQSVSSNLAWYQQKPGERPKLLIYGASTRASGVPARFSSSGSGTDFTLTINNLQAEDVGDYYCQQYYNYPPFTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 27 - protein, 238 aa, synthetic construct
METDTLLLWVLLLWVPGSTGEIVMTQSPGSLAGSAGESVSINCKSSQSVSSNLAWYQQKPGERPKLLIYGASTRASGVPARFSSSGSGTDFTLTINNLQAEDVGDYYCQQYYNFPPFTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 28 - protein, 348 aa, synthetic construct
MGWSCIILFLVATATGVHSVLPAKPENISCIFYYEENFTCTWSPEKEASYTWYKVKRTYSYGYKSDICSTDNSTRGNHASCSFLPPTITNPDNYTIQVEAQNADGIMKSDITYWNLDAIMKIEPPEIFSVKSVLGIKRMLQIKWIRPVLAPHSSTLKYTLRFRTINSAYWMEVNFTKEDIDRDETYNLTELQAFTEYVMTLRCAPAESMFWSGWSQEKVGTTEEEAPYGLDLWRVLKPAMVDGRRPVQLMWKKATGAPVLEKALGYNIWYFPENNTNLTETVNTTNQTHELYLGGKTYWVYVVSYNSLGESPVATLRIPALNEKTGLNDIFEAQKIEWHEHHHHHHHH

SEQ ID NO: 29 - protein, 8 aa, synthetic construct
GFTFDDYG

SEQ ID NO: 30 - protein, 7 aa, synthetic construct
INWAGGT

SEQ ID NO: 31 - protein, 23 aa, synthetic construct
ARESPLRLGDLGGDYFYYYYYMD

SEQ ID NO: 32 - protein, 7 aa, synthetic construct
QSVSSRY

SEQ ID NO: 33 - protein, 9 aa, synthetic construct
QQYDNSPRT

SEQ ID NO: 34 - protein, 8 aa, synthetic construct
GFTFSSYA

SEQ ID NO: 35 - protein, 8 aa, synthetic construct
ISGSGNST

SEQ ID NO: 36 - protein, 11 aa, synthetic construct
ATQVVYYFKMD

SEQ ID NO: 37 - protein, 6 aa, synthetic construct
QSVSSN

SEQ ID NO: 38 - protein, 10 aa, synthetic construct
QQYYNWPPFT

SEQ ID NO: 39 - protein, 7 aa, synthetic construct
INYAGGT

SEQ ID NO: 40 - protein, 7 aa, synthetic construct
INFAGGT

SEQ ID NO: 41 - protein, 23 aa, synthetic construct
ARESPLRLGDLGGDYFYYYYYLD

SEQ ID NO: 42 - protein, 23 aa, synthetic construct
ARESPLRLGDLGGDYFYYYYYID

SEQ ID NO: 43 - protein, 9 aa, synthetic construct
QQYDQSPRT

SEQ ID NO: 44 - protein, 9 aa, synthetic construct
QQYDNAPRT

SEQ ID NO: 45 - protein, 8 aa, synthetic construct
ISGSGQST

SEQ ID NO: 46 - protein, 8 aa, synthetic construct
ISGSGNAT

SEQ ID NO: 47 - protein, 12 aa, synthetic construct
ATQVVYYFKLDV

SEQ ID NO: 48 - protein, 12 aa, synthetic construct
ATQVVYYFKIDV

SEQ ID NO: 49 - protein, 10 aa, synthetic construct
QQYYNYPPFT

SEQ ID NO: 50 - protein, 10 aa, synthetic construct
QQYYNFPPFT

SEQ ID NO: 51 - protein, 15 aa, synthetic construct
GLNDIFEAQKIEWHE

SEQ ID NO: 52 - protein, 8 aa, synthetic construct
HHHHHHHH

SEQ ID NO: 53 - protein, 8 aa, synthetic construct
  VARIANT 1: note = A can be replaced by G, I, L, M, W, F, P or V
  VARIANT 2: note = A can be replaced by G, I, L, M, W, F, P, V or Y
  VARIANT 3: note = C can be replaced by S, T, Y, N or Q
  VARIANT 4: note = A can be replaced by G, I, L, M, W, F, P, V or Y
  VARIANT 5: note = D can be replaced by E
  VARIANT 6: note = D can be replaced by E
  VARIANT 7: note = C can be replaced by S, T, Y, N or Q
  VARIANT 8: note = A can be replaced by G, I, L, M, W, F, P or V
AACADDCA

SEQ ID NO: 54 - protein, 7 aa, synthetic construct
  VARIANT 1: note = A can be replaced by G, I, L, M, W, F, P or V
  VARIANT 2: note = C can be replaced by S, T, Y, N or Q
  VARIANT 3: note = A can be replaced by I, L, M, F, P, W, Y or V
  VARIANT 4: note = A can be replaced by G, I, L, M, W, F, P or V
  VARIANT 5: note = A can be replaced by G, I, L, M, W, F, P or V
  VARIANT 6: note = A can be replaced by G, I, L, M, W, F, P or V
  VARIANT 7: note = C can be replaced by S, T, Y, N or Q
ACAAAAC

SEQ ID NO: 55 - protein, 23 aa, synthetic construct
  VARIANT 1: note = A can be replaced by G, I, L, M, W, F, P or V
  VARIANT 2: note = H can be replaced by K or R
  VARIANT 3: note = E can be replaced by D
  VARIANT 4: note = C can be replaced by S, T, Y, N or Q
  VARIANT 5: note = A can be replaced by G, I, L, M, W, F, P or V
  VARIANT 6: note = A can be replaced by G, I, L, M, W, F, P or V
  VARIANT 7: note = H can be replaced by K or R
  VARIANT 8: note = A can be replaced by G, I, L, M, W, F, P or V
  VARIANT 9: note = A can be replaced by G, I, L, M, W, F, P or V
  VARIANT 10: note = E can be replaced by D
  VARIANT 11: note = A can be replaced by G, I, L, M, W, F, P or V
  VARIANT 12: note = A can be replaced by G, I, L, M, W, F, P or V
  VARIANT 13: note = A can be replaced by G, I, L, M, W, F, P or V
  VARIANT 14: note = E can be replaced by D
  VARIANT 15: note = C can be replaced by S, T, Y, N or Q
  VARIANT 16: note = A can be replaced by G, I, L, M, W, F, P, V or Y
  VARIANT 17: note = C can be replaced by S, T, Y, N or Q
  VARIANT 18: note = C can be replaced by S, T, Y, N or Q
  VARIANT 19: note = C can be replaced by S, T, Y, N or Q
  VARIANT 20: note = C can be replaced by S, T, Y, N or Q
  VARIANT 21: note = C can be replaced by S, T, Y, N or Q
  VARIANT 22: note = A can be replaced by G, I, L, M, W, F, P or V
  VARIANT 23: note = E can be replaced by D
AHECAAHAAEAAAECACCCCCAE

SEQ ID NO: 56 - protein, 7 aa, synthetic construct
  VARIANT 1: note = C can be replaced by S, T, Y, N or Q
  VARIANT 2: note = C can be replaced by S, T, Y, N or Q
  VARIANT 3: note = A can be replaced by G, I, L, M, W, F, P or V
  VARIANT 4: note = C can be replaced by S, T, Y, N or Q
  VARIANT 5: note = C can be replaced by S, T, Y, N or Q
  VARIANT 6: note = H can be replaced by K or R
  VARIANT 7: note = C can be replaced by S, T, Y, N or Q
CCACCHC

SEQ ID NO: 57 - protein, 9 aa, synthetic construct
  VARIANT 1: note = C can be replaced by S, T, Y, N or Q
  VARIANT 2: note = C can be replaced by S, T, Y, N or Q
  VARIANT 3: note = C can be replaced by S, T, Y, N or Q
  VARIANT 4: note = E can be replaced by D
  VARIANT 5: note = C can be replaced by S, T, Y, N or Q
  VARIANT 6: note = C can be replaced by L, S, T, Y, N or Q
  VARIANT 7: note = A can be replaced by G, I, L, M, W, F, P or V
  VARIANT 8: note = H can be replaced by K or R
  VARIANT 9: note = C can be replaced by S, T, Y, N or Q
CCCECCAHC

SEQ ID NO: 58 - protein, 7 aa, synthetic construct
  VARIANT 3: note = F can be replaced by W or Y
INFAGGT

SEQ ID NO: 59 - protein, 23 aa, synthetic construct
  VARIANT 22: note = I can be replaced by L or M
ARESPLRLGDLGGDYFYYYYYID

SEQ ID NO: 60 - protein, 9 aa, synthetic construct
  VARIANT 5: note = N can be replaced by Q
  VARIANT 6: note = S can be replaced by L
QQYDNSPRT

SEQ ID NO: 61 - protein, 8 aa, synthetic construct
  VARIANT 1: note = A can be replaced by G, I, L, M, W, F, P or V
  VARIANT 2: note = A can be replaced by G, I, L, M, W, F, P, V or Y
  VARIANT 3: note = C can be replaced by S, T, Y, N or Q
  VARIANT 4: note = A can be replaced by G, I, L, M, W, F, P, V or Y
  VARIANT 5: note = C can be replaced by S, T, Y, N or Q
  VARIANT 6: note = C can be replaced by S, T, Y, N or Q
  VARIANT 7: note = C can be replaced by S, T, Y, N or Q
  VARIANT 8: note = A can be replaced by G, I, L, M, W, F, P, or V
AACACCCA

SEQ ID NO: 62 - protein, 7 aa, synthetic construct
  VARIANT 1: note = A can be replaced by G, I, L, M, W, F, P or V
  VARIANT 2: note = C can be replaced by S, T, Y, N or Q
  VARIANT 3: note = A can be replaced by G, I, L, M, W, F, P or V
  VARIANT 4: note = C can be replaced by S, T, Y, N or Q
  VARIANT 5: note = A can be replaced by G, I, L, M, W, F, P or V
  VARIANT 6: note = C can be replaced by S, T, Y, N or Q
  VARIANT 7: note = A can be replaced by C, S, T, Y, N or Q
ACACACA

SEQ ID NO: 63 - protein, 11 aa, synthetic construct
  VARIANT 1: note = A can be replaced by G, I, L, M, W, F, P or V
  VARIANT 2: note = C can be replaced by S, T, Y, N or Q
  VARIANT 3: note = C can be replaced by S, T, Y, N or Q
  VARIANT 4: note = A can be replaced by G, I, L, M, W, F, P or V
  VARIANT 5: note = A can be replaced by G, I, L, M, W, F, P or V
  VARIANT 6: note = C can be replaced by S, T, Y, N or Q
  VARIANT 7: note = C can be replaced by S, T, Y, N or Q
  VARIANT 8: note = A can be replaced by G, I, L, M, W, F, P, V or Y
  VARIANT 9: note = H can be replaced by K or R
  VARIANT 10: note = A can be replaced by G, I, L, M, W, F, P or V
  VARIANT 11: note = E can be replaced by D
ACCAACCAHAE

SEQ ID NO: 64 - protein, 6 aa, synthetic construct
  VARIANT 1: note = C can be replaced by S, T, Y, N or Q
  VARIANT 2: note = C can be replaced by S, T, Y, N or Q
  VARIANT 3: note = A can be replaced by G, I, L, M, V, W, F, P or V
  VARIANT 4: note = C can be replaced by S, T, Y, N or Q
  VARIANT 5: note = C can be replaced by S, T, Y, N or Q
  VARIANT 6: note = C can be replaced by S, T, Y, N or Q
CCACCC

SEQ ID NO: 65 - protein, 10 aa, synthetic construct
  VARIANT 1: note = C can be replaced by S, T, Y, N or Q
  VARIANT 2: note = C can be replaced by S, T, Y, N or Q
  VARIANT 3: note = A can be replaced by G, I, L, M, W, F, P, V or Y
  VARIANT 4: note = A can be replaced by G, I, L, M, W, F, P, V or Y
  VARIANT 5: note = C can be replaced by S, T, Y, N or Q
  VARIANT 6: note = A can be replaced by I, L, M, F, P, W, Y or V
  VARIANT 7: note = A can be replaced by G, I, L, M, W, F, P or V
  VARIANT 8: note = A can be replaced by G, I, L, M, W, F, P or V
  VARIANT 9: note = A can be replaced by G, I, L, M, W, F, P, V or Y
  VARIANT 10: note = C can be replaced by S, T, Y, N or Q
CCAACAAAAC

SEQ ID NO: 66 - protein, 8 aa, synthetic construct
  VARIANT 6: note = N can be replaced by Q
  VARIANT 7: note = A can be replaced by S
ISGSGNAT

SEQ ID NO: 67 - protein, 12 aa, synthetic construct
  VARIANT 10: note = I can be replaced by L or M
ATQVVYYFKIDV

SEQ ID NO: 68 - protein, 10 aa, synthetic construct
  VARIANT 6: note = F can be replaced by Y or W
QQYYNFPPFT

SEQ ID NO: 69 - protein, 12 aa, synthetic construct
SGFTFDDYGMSW

SEQ ID NO: 70 - protein, 19 aa, synthetic construct
SGINWAGGTIGYADAVKGR

SEQ ID NO: 71 - protein, 26 aa, synthetic construct
CARESPLRLGDLGGDYFYYYYYMDVW

SEQ ID NO: 72 - protein, 14 aa, synthetic construct
CRASQSVSSRYLVW

SEQ ID NO: 73 - protein, 9 aa, synthetic construct
YGTSSRATG

SEQ ID NO: 74 - protein, 11 aa, synthetic construct [sequence omitted]

SEQ ID NO: 75 - protein, 19 aa, synthetic construct
SAISGSGNSTYYADAVKGR

SEQ ID NO: 76 - protein, 14 aa, synthetic construct
CATQVVYYFKMDVW

SEQ ID NO: 77 - protein, 13 aa, synthetic construct
CKSSQSVSSNLAW

SEQ ID NO: 78 - protein, 9 aa, synthetic construct
YGASTRASG

SEQ ID NO: 79 - protein, 12 aa, synthetic construct [sequence omitted]

SEQ ID NO: 80 - protein, 12 aa, synthetic construct
SGFTFSSYAMSW

SEQ ID NO: 81 - protein, 8 aa, synthetic construct
GGGSGGGS

SEQ ID NO: 82 - protein, 25 aa, synthetic construct
EVQLVESGGGVVRPGGSLRLSCAAS

SEQ ID NO: 83 - protein, 25 aa, synthetic construct
EVQLVESGGDLVKPGGSLRLSCVAS

SEQ ID NO: 84 - protein, 17 aa, synthetic construct
MSWVRQIPGRGLEWVSG

SEQ ID NO: 85 - protein, 17 aa, synthetic construct
MSWVRQAPGKGLQWVSG

SEQ ID NO: 86 - protein, 8 aa, synthetic construct
INWAGGTI

SEQ ID NO: 87 - protein, 8 aa, synthetic construct
INYAGGTI

SEQ ID NO: 88 - protein, 8 aa, synthetic construct
INFAGGTI

SEQ ID NO: 89 - protein, 38 aa, synthetic construct
GYADSVKGRFTVSRDDANNSLYLQMNSLRAEDTALYLC

SEQ ID NO: 90 - protein, 38 aa, synthetic construct
GYADAVKGRFTISRDNARNTVYLQMNSLRAEDTAVYYC

SEQ ID NO: 91-92 - skipped (2 entries)

SEQ ID NO: 93 - protein, 24 aa, synthetic construct
ARESPLRLGDLGGDYFYYYYYMDV

SEQ ID NO: 94 - protein, 24 aa, synthetic construct
ARESPLRLGDLGGDYFYYYYYLDV

SEQ ID NO: 95 - protein, 24 aa, synthetic construct
ARESPLRLGDLGGDYFYYYYYIDV

SEQ ID NO: 96 - protein, 11 aa, synthetic construct
WGKGTTVTVSS

SEQ ID NO: 97 - protein, 11 aa, synthetic construct
WGQGTLVTVSS

SEQ ID NO: 98 - protein, 26 aa, synthetic construct [sequence omitted]

SEQ ID NO: 99 - protein, 26 aa, synthetic construct
EVVMIQTPLSLSVSPGEPASISCRAS

SEQ ID NO: 100 - protein, 17 aa, synthetic construct
LVWYQQKPGQAPRLLIY

SEQ ID NO: 101 - protein, 17 aa, synthetic construct
LVWYLQKPGQSPQLLIY

SEQ ID NO: 102 - protein, 36 aa, synthetic construct [sequence omitted]

SEQ ID NO: 103 - protein, 36 aa, synthetic construct
SRATGVSDRFSGSGSGTDFTLRISRVEAEDVGVYYC

SEQ ID NO: 104 - protein, 10 aa, synthetic construct
FGQGTKVEIK

SEQ ID NO: 105 - protein, 25 aa, synthetic construct
EVQLLESGGGLIQPGGSLRLSCGAS

SEQ ID NO: 106 - protein, 17 aa, synthetic construct
MSWVRQAPGKGLEWVSA

SEQ ID NO: 107 - protein, 17 aa, synthetic construct
MSWVRQAPGKGLQWVSA

SEQ ID NO: 108 - protein, 38 aa, synthetic construct
YYADSVKGRFTISRDNSKNTLYLQMNSLRAEDTAVYYC

SEQ ID NO: 109 - protein, 38 aa, synthetic construct
YYADAVKGRFTISRDNAKNTLYLQMNSLRAEDTAVYYC

SEQ ID NO: 110 - protein, 26 aa, synthetic construct
EIVMTQSPATLSVSPGERATLSCRAS

SEQ ID NO: 111 - protein, 26 aa, synthetic construct
EIVMTQSPGSLAGSAGESVSINCKSS

SEQ ID NO: 112 - protein, 17 aa, synthetic construct
LAWYRQKPGQAPRLLIY

SEQ ID NO: 113 - protein, 17 aa, synthetic construct
LAWYQQKPGERPKLLIY

SEQ ID NO: 114 - protein, 36 aa, synthetic construct
TRATGIPARFSGSGSGTEFTLTISSLQSEDFAVYYC

SEQ ID NO: 115 - protein, 36 aa, synthetic construct
TRASGVPARFSSSGSGTDFTLTINNLQAEDVGDYYC

SEQ ID NO: 116 - protein, 10 aa, synthetic construct
FGPGTKLDIK

SEQ ID NO: 117 - protein, 10 aa, synthetic construct
FGQGTKLEIK